(19) United States
(12) Patent Application Publication
(10) Pub. No.: US 2025/0049835 A1
(43) Pub. Date: Feb. 13, 2025

(54) COMPOSITIONS FOR ALTERING CYCLIC ADP-RIBOSE SECONDARY MESSENGER SIGNALLING

(71) Applicants: Yeda Research and Development Co. Ltd., Rehovot (IL); Dana-Farber Cancer Institute, Inc., Boston, MA (US)

(72) Inventors: Rotem SOREK, Rehovot (IL); Gil AMITAI, Rehovot (IL); Azita LEAVITT, Rehovot (IL); Erez YIRMIYA, Rehovot (IL); Philip J. KRANZUSCH, Somerville, MA (US); Allen LU, Boston, MA (US); Samuel J. HOBBS, Boston, MA (US)

(73) Assignees: Yeda Research and Development Co. Ltd., Rehovot (IL); Dana-Farber Cancer Institute, Inc., Boston, MA (US)

(21) Appl. No.: 18/926,385

(22) Filed: Oct. 25, 2024

Related U.S. Application Data
(63) Continuation of application No. PCT/IL2023/050418, filed on Apr. 24, 2023.
(60) Provisional application No. 63/334,217, filed on Apr. 25, 2022.
(60) Provisional application No. 63/442,552, filed on Feb. 1, 2023.

(51) Int. Cl. A61K 31/7076; A61K 39/39; A61K 39/395

(52) U.S. Cl. CPC A61K 31/7076; A61K 39/39; A61K 39/3955

(57) ABSTRACT

Methods of treating a disease or disorder associated with a down or up-regulation of an immune response of a subject are disclosed. The methods comprising administering to the subject a therapeutically effective amount of any one or more of the molecule 1′-2′ glycosyl cyclic adenosine diphosphate ribose (1′-2′ gcADPR), 1′-3′ gcADPR or Tad1 protein.

### RELATED APPLICATIONS

This application is a Continuation of PCT Patent Application No. PCT/IL2023/050418 having International filing date of Apr. 24, 2023, which claims the benefit of priority under 35 USC § 119 (e) of U.S. Provisional Patent Application Nos. 63/334,217 filed on Apr. 25, 2022 and 63/442,552 filed on Feb. 1, 2023. The contents of the above applications are all incorporated by reference as if fully set forth herein in their entirety.

### SEQUENCE LISTING STATEMENT

The XML file, entitled 101469SequenceListing.xml, created on Oct. 23, 2024, comprising 40,827 bytes, submitted concurrently with the filing of this application is incorporated herein by reference.

### FIELD AND BACKGROUND OF THE INVENTION

The present invention, in some embodiments thereof, relates to immune modulating agents and methods of treating diseases or disorders associated with a down-regulated or an up-regulated immune response using same. The present invention also relates to agents and methods for treating neuronal damage.

The Toll/interleukin-1 receptor (TIR) domain serves as the signal transducing module in immune receptors that recognize pathogen invasion in the immune systems of bacteria, plants, and animals. Whereas TIR domains in animals mainly transfer the signal by protein-protein interactions, in plants and bacteria these domains produce an immune signaling molecule, which has the same mass as cyclic ADP-ribose (cADPR), but whose molecular and chemical structure remains elusive.

The mechanism of action of TIR-mediated immune signaling was recently deciphered for a bacterial anti-phage immune system called Thoeris¹. This system comprises two core proteins, one of which (named ThsB) has a TIR domain and serves as the sensor for phage infection. Recognition of phage triggers the ThsB TIR domain to produce the cADPR isomer molecule, and this molecule activates a second Thoeris protein, ThsA, which then depletes the cell of the essential molecule nicotinamide adenine dinucleotide (NAD⁺) and leads to premature cell death to abort the infection^{1.6}. Intriguingly, activation of plant TIRs by pathogens also leads to cell suicide that prevents pathogen propagation, and it was hypothesized that the elusive cADPR isomer produced by the TIRs is involved in mediating this plant immune response^{4.5.7}.

The immune signaling molecule 2′3′-cyclic GMP-AMP (cGAMP) plays a role in numerous immune-related disorders and up-regulation of this molecule has been shown to be effective for treating a wide range of diseases including cancer, whilst down-regulation (or blockage) of the molecule has been proposed for the treatment of autoimmune diseases.

Identification of novel immune signaling molecules may thus be of relevance in the search for new therapeutics in the treatment of immune related diseases.

Background art includes Ofir, G. et al. Nature 600, 116-120 (2021).

### SUMMARY OF THE INVENTION

According to an aspect of the present invention there is provided a method of treating a disease or disorder associated with a down-regulation of an immune response of a subject comprising administering to the subject a therapeutically effective amount of any one or more of the molecules 1′-2′ glycosyl cyclic adenosine diphosphate ribose (1′-2′ gcADPR), and 1′-3′ glycosyl cyclic adenosine diphosphate ribose (1′-3′ gcADPR), or a salt, an enantiomer, solvate or hydrate thereof, thereby treating the disease or disorder associated with a down-regulation of an immune response of a subject.

The terms 1′-2′ gcADPR and 1″-2′ gcADPR are interchangeable. The terms 1′-3′ gcADPR and 1″-3′ gcADPR are interchangeable.

According to embodiments of the invention, the disease or disorder associated with a down-regulation of the immune response is cancer.

According to embodiments of the invention, the cancer is selected from the group consisting of melanoma, breast cancer, pancreatic cancer, prostate cancer, lung cancer, colorectal cancer.

According to embodiments of the invention, the 1′-2′ gcADPR is comprised in bacteria.

According to embodiments of the invention, the 1′-3′ gcADPR is comprised in bacteria.

According to embodiments of the invention, 1′-2′ gcADPR and 1′-3′ gcADPR are comprised in bacteria.

According to embodiments of the invention, the administering comprises intratumoral administration.

According to embodiments of the invention, the method further comprises administering to the subject a therapeutically effective amount of an antibody directed against an immune checkpoint protein.

According to embodiments of the invention, the antibody is anti-PD1 antibody or an anti-OX40 antibody.

According to an aspect of the present invention there is provided an article of manufacture comprising:
(i) at least one of 1′-2′ gcADPR and/or 1′-3′ gcADPR, or a salt, an enantiomer, solvate or hydrate thereof; and
(ii) an antibody directed against an immune checkpoint protein.

According to embodiments of the invention, the at least one of 1′-2′ gcADPR and/or 1′-3′ gcADPR, and the antibody are formulated in a single composition.

According to an aspect of the present invention there is provided a vaccine comprising a disease associated antigen and an adjuvant comprising at least one of 1′-2′ gcADPR and/or 1′-3′ gcADPR, or a salt, an enantiomer, solvate or hydrate thereof.

According to an aspect of the present invention there is provided a method of treating an autoimmune disease or a disease associated with neuronal degeneration in a subject in need thereof comprising administering to the subject a therapeutically effective amount of a Thoeris anti-defense 1 (Tad1) polypeptide having an amino acid sequence at least 80% identical to any one of SEQ ID NOs: 1-11, or a polynucleotide encoding the Tad1 polypeptide, the Tad1 polypeptide comprising a 1′-2′ gcADPR and/or a 1′-3′ gcADPR sequestering activity, thereby treating the autoimmune disease or the disease associated with neuronal degeneration.

According to embodiments of the invention, the Tad1 polypeptide comprises an amino acid sequence at least 90% identical to SEQ ID NO: 1.

According to embodiments of the invention, the method is for treating a neuronal injury in the subject.

According to embodiments of the invention, the neuronal injury is brought about by a neurodegenerative disease, stroke, a traumatic brain injury, a spinal cord injury, a peripheral nerve injury or an eye injury.

According to embodiments of the invention, the neurodegenerative disease is selected from the group consisting of Amyotrophic Lateral Sclerosis (ALS), Parkinson's disease, Multiple System Atrophy (MSA), Huntington's disease, Alzheimer's disease, Rett Syndrome and Multiple Sclerosis (MS).

According to an aspect of the present invention there is provided a method of screening for an agent that alters immune modulation comprising:
contacting the agent with a ThsA enzyme;
measuring the NADase activity of the ThsA enzyme wherein a change in NADase activity is indicative of an agent that alters immune modulation.

According to embodiments of the invention, the contacting is effected in the presence of at least one of 1′-2′ gcADPR and/or 1′-3′ gcADPR.

According to embodiments of the invention, the contacting is effected in the presence of a Tad1 polypeptide.

According to embodiments of the invention, the ThsA enzyme is Bacillus cereus MSX-D12 ThsA enzyme.

According to an aspect of the present invention there is provided a method of isolating 1′-2′ gcADPR comprising:
(a) contacting the 1′-2′ gcADPR comprised in bacterial cells or cell lysates generated therefrom, with a Tad1 polypeptide under conditions which generate a complex between the 1′-2′ gcADPR and the Tad1 polypeptide; and
(b) isolating the complex using an agent that specifically binds to the Tad1 polypeptide.

According to an aspect of the present invention there is provided a method of isolating 1′-3′ gcADPR comprising:
(a) contacting the 1′-3′ gcADPR comprised in bacterial cells or cell lysates generated therefrom, with a Tad1 polypeptide under conditions which generate a complex between the 1′-3′ gcADPR and the Tad1 polypeptide; and
(b) isolating the complex using an agent that specifically binds to the Tad1 polypeptide.

According to embodiments of the invention, the Tad1 polypeptide comprises a separating moiety.

According to embodiments of the invention, the Tad1 polypeptide is co-expressed with a Toll/interleukin-1 receptor (TIR) domain protein in the bacterial cells to generate the 1′-2′ gcADPR and/or the 1′-3′ gcADPR.

According to embodiments of the invention, the TIR domain protein is Brachypodium distachyon TIR domain protein.

According to embodiments of the invention, the bacterial cells are E. coli cells.

According to embodiments of the invention, the 1′-2′ gcADPR is generated by activating ThsB of the bacterial cells.

According to embodiments of the invention, the 1′-3′ gcADPR is generated by activating ThsB of the bacterial cells.

According to embodiments of the invention, the Tad1 polypeptide has an amino acid sequence as set forth in any one of SEQ ID NOs: 1-10.

According to embodiments of the invention, the activating comprises infecting the bacterial cells with a bacteriophage that do not express Tad1.

According to an aspect of the present invention there is provided a method of producing gcADPR comprising isolating gcADPR from a composition comprising Toll/interleukin-1 receptor (TIR) domain protein and β-NAD+.

According to embodiments of the invention, said TIR domain protein comprises AaTIR

(MKNRSYEYDV ALSFAGENRA YVERVANSLK TKGVKVFYDL FEEANLWGKN LYEYLSEIYQ
NKARYTVLFV SSFYNKKLWT NHERVSMQAR AFQESREYIL PARFDDTEIP GILKTIGYIN
LENRTPEELA VLIENKLKKD QTFF; SEQ ID NO: 28)
and
AbTIR
(MEYDLFISHA SEDKEDFVRP LAETLQQLGV NVWYDEFTLK VGDSLRQKID SGLRNSKYGT
VVLSTDFIKK DWTNYELDGL VAREMNGHKM ILPIWHKITK NDVLDYSPNL ADKVALNTSV
NSIEEIAHQL ADVILNR; SEQ ID NO: 29).

According to embodiments of the invention said TIR domain protein is consisting of AaTIR (SEQ ID NO: 28) and AbTIR (SEQ ID NO: 29).

According to embodiments of the invention the method is performed in the absence of Tad1.

According to embodiments of the invention said gcAPDR comprises at least one of 1′-2′ gcADPR and 1′-3′ gcADPR.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D illustrate that Tad1 inhibits Theoris defense. (A) Genome comparison of eight phages from the SBSphiJ group. Amino acid sequence similarity between the ORFs is marked by grey shading. Genome similarity was visualized using clinker¹⁰. (B) Differential defense of Theoris against SBSphiJ phages, and anti-Theoris activity of Tad1. Data represent plaque-forming units per ml (PFU/mL) of phages infecting control cells (“no system”), cells expressing the Thoeris system (“Thoeris”), and cells co-expressing the Theoris system and the tad1 gene from SBSphiJ7. All phages except for SBSphiJ7 lack tad1. Shown is the average of three replicates, with individual data points overlaid. (C) The tad1 locus in SBSphiJ7. Shown in the locus for two other phages, SBSphiJ3 and SBSphiJ4, in which tad1 is absent. The coordinates of the presented locus within the phage genome are indicated below the name of each phage. (D) Tad1 knockdown cancels anti-Theoris activity. Results of phage SBSphiJ7 infection experiments. Data represent PFU/mL of SBSphiJ7 infecting cells expressing Theoris and a dCas9 system targeting Tad1, as well as control cells. Shown is the average of three replicates, with individual data points overlaid;

FIGS. 2A-B illustrate that Tad1 proteins inhibit Theoris defense. (A) Tad1 homologs can inhibit the Thoeris system in B. subtilis. Shown are tenfold serial dilution plaque assays with phage SBSphiJ. (B) Results of phage infection experiments with eight phages of the SBSphiJ family. Data represent PFU/mL of phages infecting control cells (“no system”), cells expressing the Thoeris system (“Thoeris”), and cells co-expressing the Theoris system and a Tad1 homolog. All phages except for SBSphiJ7 lack Tad1. Shown is the average of three replicates, with individual data points overlaid. The “Thoeris” and “no system” data presented here are the same as those presented in FIG. 1B;

FIGS. 3A-G illustrate that Tad1 inhibits Theoris-mediated defense by physically binding and sequestering the Theoris-derived signaling molecule. (A) Schematic representation of the ThsB/Tad1 co-expression experiment. Cells expressing ThsB, both ThsB and Tad1, or control cells were infected with phage SBSphiJ. NADase activity of ThsA incubated with filtered lysates was measured using a nicotinamide 1,N6-ethenoadenine dinucleotide (&NAD) cleavage fluorescence assay. (B) Activation of ThsA by lysates from infected cells. NADase activity of purified ThsA protein incubated with filtered lysates derived from cells expressing ThsB (native promoter), cells expressing, in addition to ThsB, also Tad1 from SBSphiJ7 (induced by 1 mM IPTG), or control cells that do not express ThsB, that were infected by phage SBSphiJ at MOI of 5. Bars represent the mean of three experiments, with individual data points overlaid. (C) Co-expression of Tad1 (induced by 1 mM IPTG) with ThsB (native promoter) eliminates the signaling molecule normally produced by ThsB in infected cells. Cells were infected with SBSphiJ at an MOI of 5, and lysates were analyzed by LC-MS. Y axis represents the area under curve (AUC) of cADPR isomer ions detected in the MS analysis. Time 0 represents uninfected cells. (D) Schematic representation of the Tad1/lysate incubation experiment. Cells expressing ThsB protein or control cells were infected with phage SBSphiJ at an MOI of 5 in 25° C. After 120 minutes the cells were lysed and lysates were filtered, and then incubated with purified cmTad1. ThsA was added to the lysates and NADasc activity of ThsA was measured. (E) Purified Tad1 eliminates the signaling molecule from infected lysates. Shown is NADase activity of purified ThsA incubated with filtered lysates derived from infected cells overexpressing ThsB (0.1 mM IPTG). Filtered lysates were either pre-incubated with 600 nM purified cmTad1 for 10 minutes in vitro (“with cmTad1”) or with buffer (“W/O cmTad1”). Control are filtered lysates from infected cells not expressing ThsB. (F) Tad1 is not an enzyme. Shown is NADase activity of purified ThsA incubated with filtered lysates as in panel B. Filtered lysates were incubated in vitro for 10, 20 or 40 minutes with purified cmTad1, or with a buffer (“W/O cmTad1”), prior to exposure to ThsA. Control is as in panel B. (G) Tad1 releases chelated molecule when denatured. Shown is NADase activity of purified ThsA incubated with filtered lysates derived from infected cells overexpressing ThsB, that were additionally pre-incubated with purified cmTad1 in vitro for 10 minutes, followed by an additional incubation of 5 minutes at cither 25° C. or 85° C. (denaturing conditions). “With cmTad1”, lysates pre-incubated with cmTad1. “W/O cmTad1”, lysates incubated with buffer instead of cmTad1. Control are lysates derived from infected cells that do not express ThsB;

FIGS. 4A-G illustrate the structure of Tad1 and identification of 1′-2′ gcADPR. (A) Overview of the cbTad1 crystal structure in front view bound to 1′-2′ gcADPR (yellow). Tad1 forms a homodimer with two ligand binding sites, with one monomer shown in orange and the other in grey. (B) Topology map of Tad1 from a top view perspective. Loops that form one binding site are highlighted in green. (C) Comparison of the ligand binding site of cbTad1 in the apo state (cyan) and in complex with the Theoris signal (orange and grey). Upon binding ligand, cbTad1 loop β4-α1 shifts ˜3.7 Å (measured by A56 amine movement) and the C-terminal tail becomes structured to enclose around the molecule. (D) Polder omit map of the Tad1 ligand-binding site contoured at 60 reveals the chemical structure of the ligand as 1′-2′ gcADPR. (E) Detailed view of cbTad1 residues interacting with the adenine base of 1′-2′ gcADPR or (F) with the ribose moieties and phosphates. Conserved residues in cmTad1 are labeled separately in parentheses. Separate monomer chains are shown in either orange or grey. Green dashed lines denote hydrogen bonding interactions. Light blue mesh denotes the 1′-2′ gcADPR 2F.-Fr electron density contoured at 1.80. (G) A model of the mechanism of Tad1;

FIG. 5 is a graph illustrating that lysates derived from cells overexpressing the TIR domain from human SARM1 (hSARM) and drosophila SARM1 (dSARM), as well as lysates derived from cells overexpressing BdTIR, activate ThsA;

FIG. 6 exhibits the results (bands) of electrophoresis separation (SDS-PAGE) corresponding to the molecular weights of the expected fractions of AaTIR and AbTIR obtained from the purification process of cell expressing AaTIR and AbTIR; and

FIGS. 7A-B are graphs representing HPLC peaks each representing a molecule obtained from the AaTIR reaction (7A, peaks a-f) and the AbTIR reaction (7B, peaks a-c).

### DESCRIPTION OF SPECIFIC EMBODIMENTS OF THE INVENTION

The present invention, in some embodiments thereof, relates to immune modulating agents and methods of treating diseases or disorders associated with a down-regulated or an up-regulated immune response using same. The present invention also relates to agents and methods for treating neuronal damage.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The Toll/interleukin-1 receptor (TIR) domain is a key component of immune receptors that identify pathogen invasion in bacteria, plants, and animals. In the antiphage defense system Thoeris, as well as in plants, recognition of infection stimulates TIR domains to produce an immune signaling molecule whose molecular structure remained elusive. This molecule binds and activates the Theoris immune effector, which then executes the immune function.

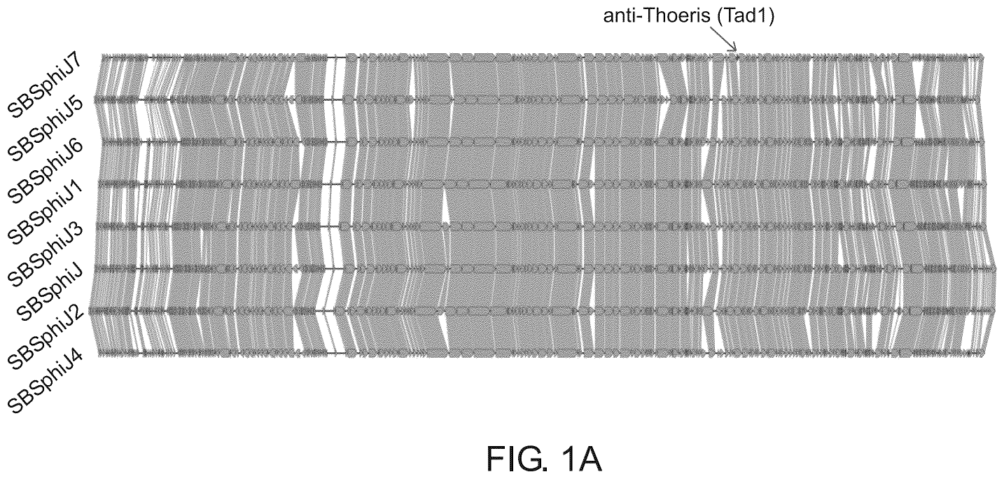

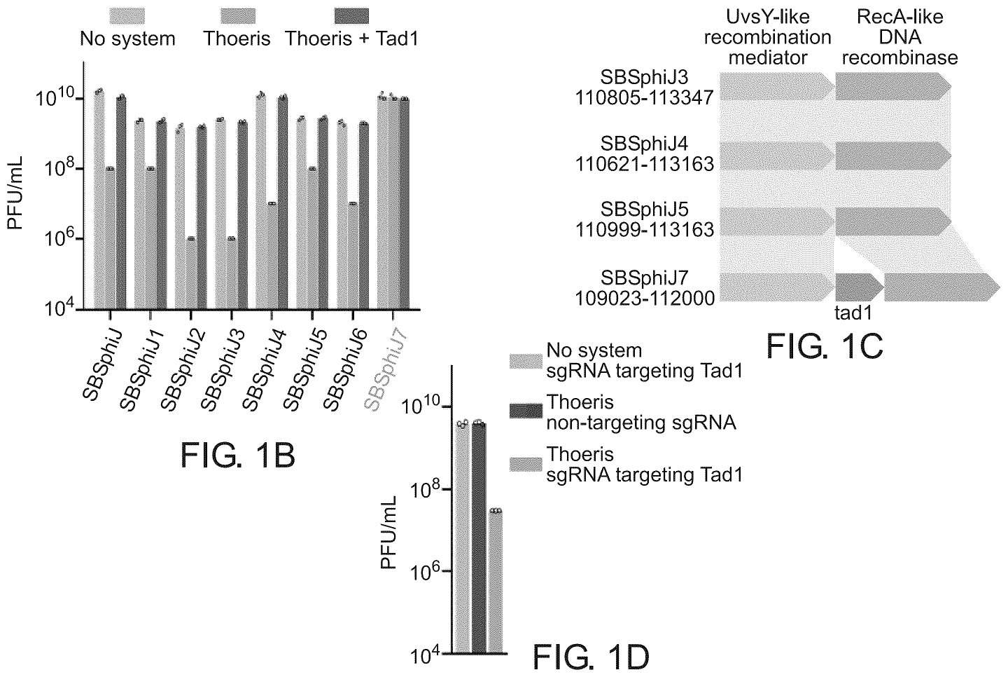

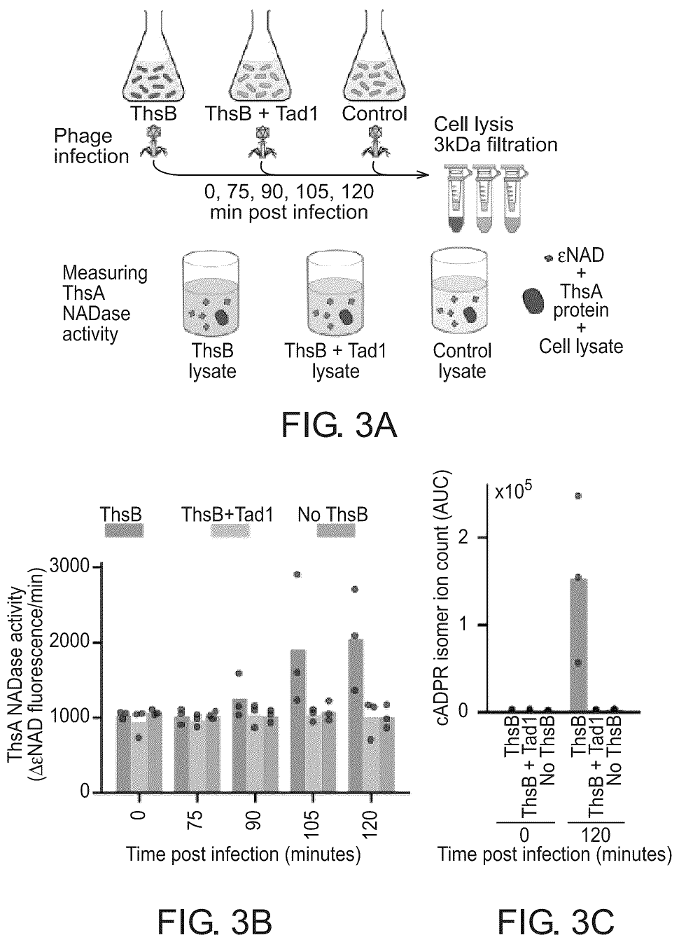

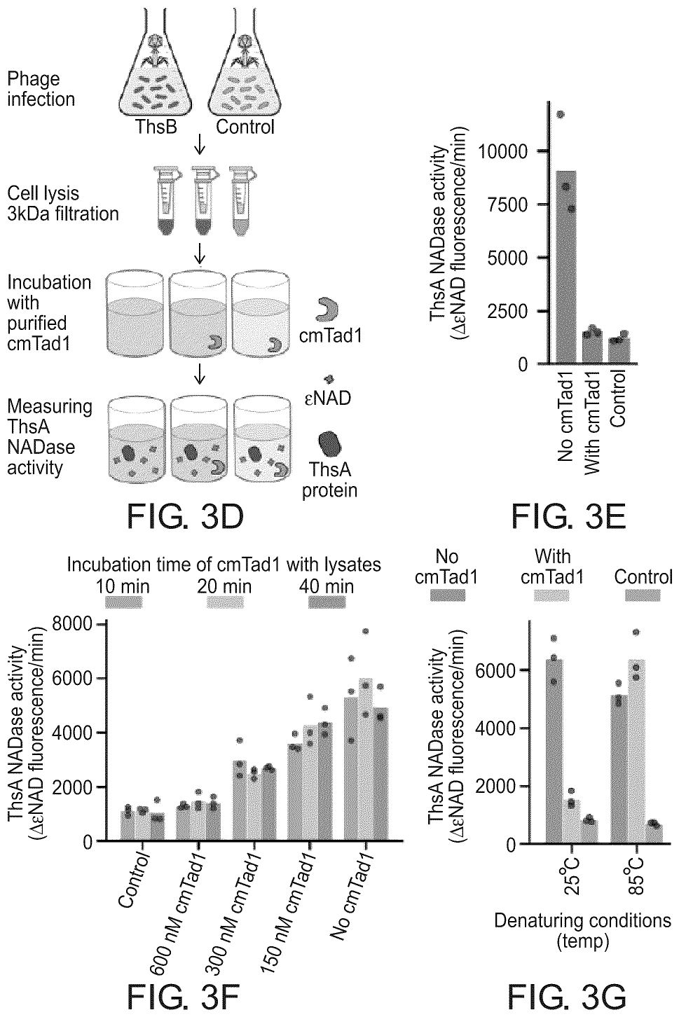

The present inventors have now identified a large family of phage-encoded proteins, denoted here Theoris anti-defense 1 (Tad1), that inhibit Thoeris immunity (see FIG. 1A). The present inventors found that Tad1 proteins are chelators (“sponges”) that bind and sequester the immune signaling molecule produced by TIR-domain proteins, thus decoupling phage sensing from immune effector activation and rendering Thoeris inactive (FIGS. 3A-G).

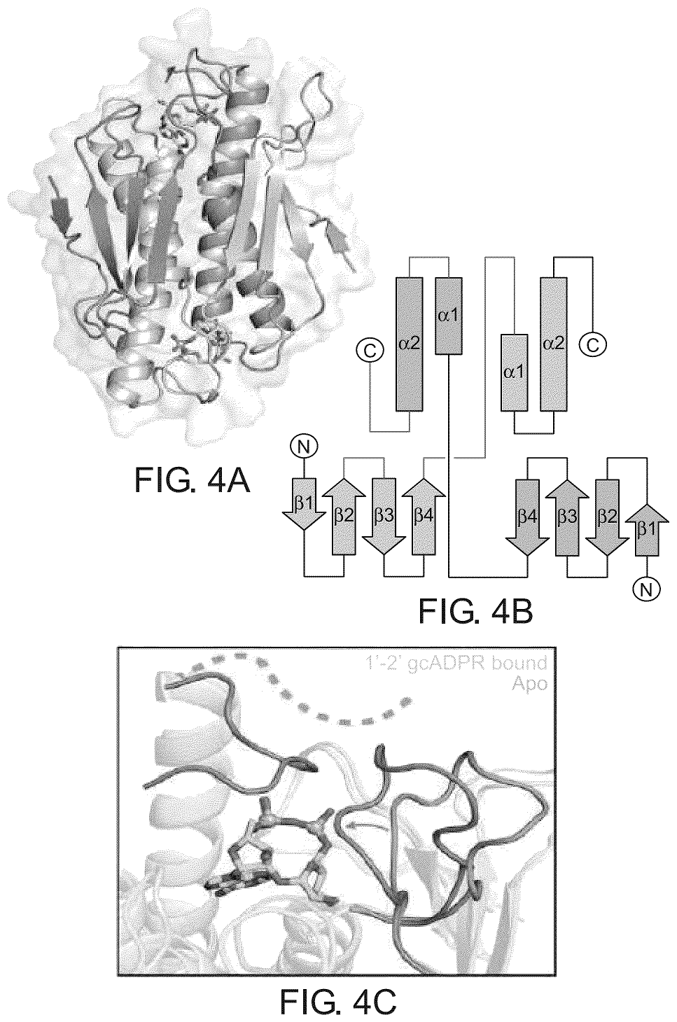

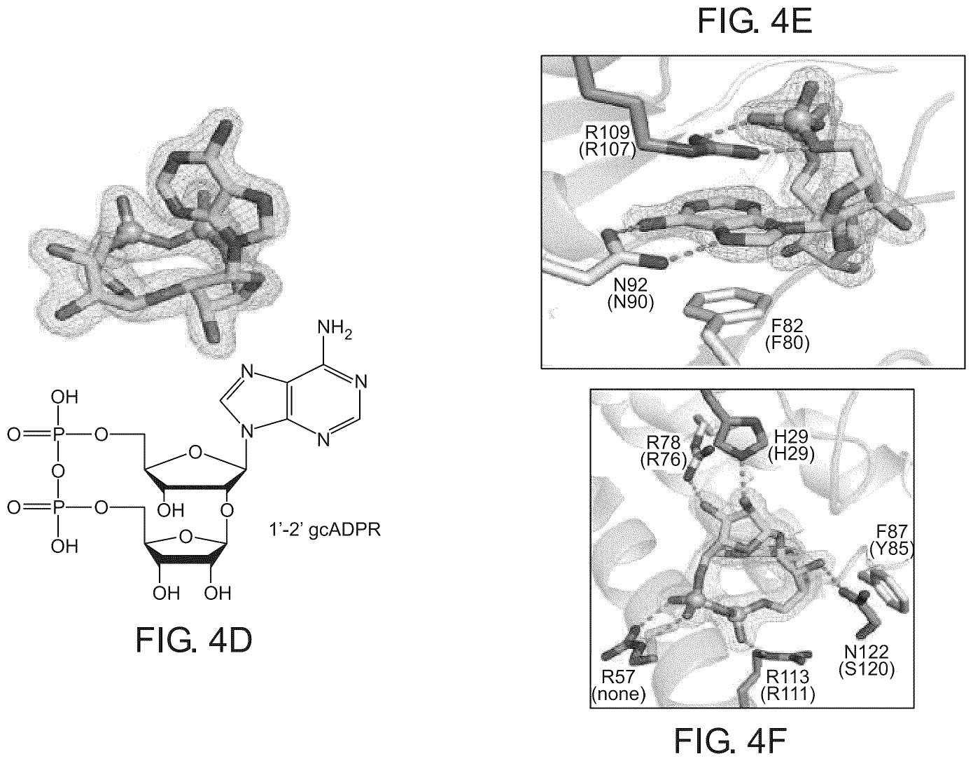

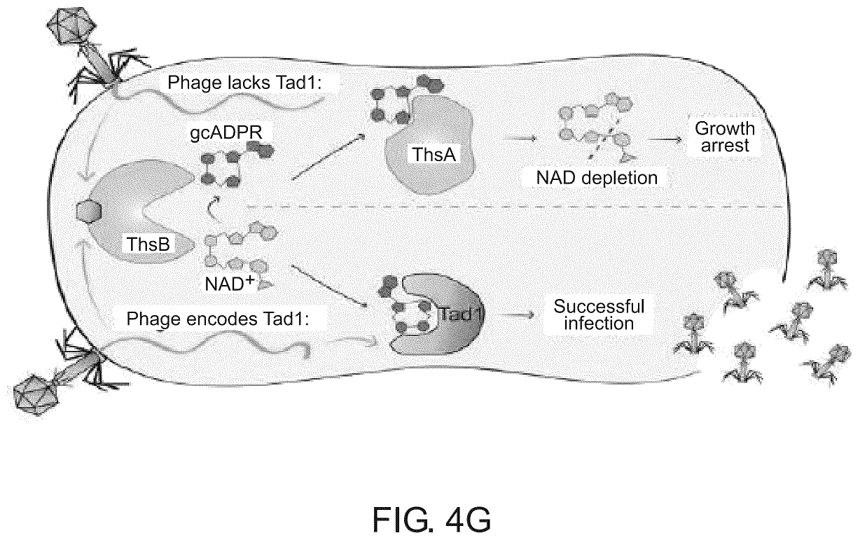

A high-resolution crystal structure of Tad1 bound to the signaling molecule revealed that its chemical structure is 1′-2′ glycocyclic ADPR (1′-2′ gcADPR), as illustrated in FIGS. 4A-F, a unique molecule not previously described in other biological systems. These results define the chemical structure of a central immune signaling molecule in bacteria and plants, and reveal a new mode of action by which pathogens can suppress host immunity.

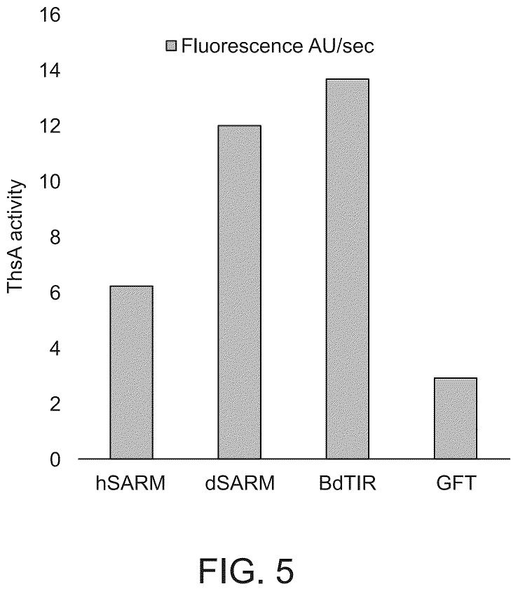

Whilst further reducing the present invention to practice, the present inventors expressed the TIR domains of both a human and drosophila TIR-domain protein in bacterial cells. This protein (Sterile alpha and TIR motif containing 1 (SARM1)) is expressed in neurons, where it plays a role in neuron degeneration in response to neuron injury and also in immune cells. As illustrated in FIG. 5, lysates derived from cells overexpressing the TIR domains of human SARM1 and drosophila SARM1 activate ThsA in vitro in a similar way that the plant TIR-domain protein (BdTIR) activates ThsA, suggesting that human TIR-domain proteins also produce 1′-2′ gcADPR and plays a role in immune modulation and neuronal processes.

Thus, according to a first aspect of the present invention, there is provided a method of treating a disease or disorder associated with a down-regulation of an immune response of a subject comprising administering to the subject a therapeutically effective amount of any one or more of the molecules 1′-2′ glycosyl cyclic adenosine diphosphate ribose (1′-2′ gcADPR) and 1′-3′ glycosyl cyclic adenosine diphosphate ribose (1′-3′ gcADPR), or a salt, an enantiomer, solvate or hydrate thereof, thereby treating the disease or disorder associated with a down-regulation of an immune response.

The term “treating” refers to inhibiting or arresting the development of a pathology (disease, injury, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings, male or female, at any age or gender, which suffer from the pathology.

The signaling molecule 1′-2′ glycosyl cyclic adenosine diphosphate ribose (hereinafter 1′-2′ gcADPR), represented by IUPAC nomenclature 1S,3R,4R,6R,14R,15S,16R,18R)-4-(6-amino-9H-purin-9-yl)-9,11,15,16,18-pentahydroxy-2,5,8,10,12,17-hexaoxa-9,11-diphosphatricyclo[12.2.1.13,6]octadecane 9,11-dioxide, is represented by the chemical structure:

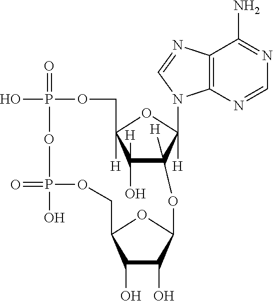 [SMILES: Nc1ncnc2c1ncn2[C@@H]1O[C@@H]2COP(=O)(O)OP(=O)(O)OC[C@H]3O[C@@H](OC1[C@H]2O)C(O)[C@H]3O]

The Thoeris molecule 1″-3′ gcADPR is an isomer of 1″-2′ gcADPR, in which the ribose-ribose glycosyl bond occurs on the 3′ carbon position of the adenine ribose.

Diseases or disorders associated with a down-regulation of an immune response include for example Severe combined immunodeficiency (SCID), a temporary acquired immune deficiency (for example after taking chemotherapeutic agent or other agents known to depress the immune system), HIV.

Since cancer cells are known to evade the immune system, another disease associated with a down-regulation of an immune response is cancer.

According to this embodiment, the subject who is treated is typically one that has been diagnosed as having cancer. In some embodiments, the cancer patient is a patient diagnosed with cancer on the basis of imaging, biopsy, staging, etc. In one embodiment, the subject typically exhibits suspicious clinical signs of lung cancer or cancer in general (e.g., persistent cough, hemoptysis, chest pain, shortness of breath, pleural effusion, wheezing, hoarseness, recurrent bronchitis or pneumonia, bone pain, paraneoplastic syndromes, unexplained pain, sweating, unexplained fever, unexplained loss of weight up to anorexia, anemia and/or general weakness).

Exemplary cancers for which 1′-2′ gcADPR and/or 1″-3′ gcADPR (or a salt, an enantiomer, solvate or hydrate thereof) is indicated include, but are not limited to, adrenocortical carcinoma, hereditary; bladder cancer; breast cancer; breast cancer, ductal; breast cancer, invasive intraductal; breast cancer, sporadic; breast cancer, susceptibility to; breast cancer, type 4; breast cancer, type 4; breast cancer-1; breast cancer-3; breast-ovarian cancer; Burkitt's lymphoma; cervical carcinoma; colorectal adenoma; colorectal cancer; colorectal cancer, hereditary nonpolyposis, type 1; colorectal cancer, hereditary nonpolyposis, type 2; colorectal cancer, hereditary nonpolyposis, type 3; colorectal cancer, hereditary nonpolyposis, type 6; colorectal cancer, hereditary nonpolyposis, type 7; dermatofibrosarcoma protuberans; endometrial carcinoma; esophageal cancer; gastric cancer, fibrosarcoma, glioblastoma multiforme; glomus tumors, multiple; hepatoblastoma; hepatocellular cancer; hepatocellular carcinoma; leukemia, acute lymphoblastic; leukemia, acute myeloid; leukemia, acute myeloid, with cosinophilia; leukemia, acute nonlymphocytic; leukemia, chronic myeloid; Li-Fraumeni syndrome; liposarcoma, lung cancer; lung cancer, small cell; lymphoma, non-Hodgkin's; lynch cancer family syndrome II; male germ cell tumor; mast cell leukemia; medullary thyroid; medulloblastoma; melanoma, meningioma; multiple endocrine neoplasia; myeloid malignancy, predisposition to; myxosarcoma, neuroblastoma; osteosarcoma; ovarian cancer; ovarian cancer, serous; ovarian carcinoma; ovarian sex cord tumors; pancreatic cancer; pancreatic endocrine tumors; paraganglioma, familial nonchromaffin; pilomatricoma; pituitary tumor, invasive; prostate adenocarcinoma; prostate cancer; renal cell carcinoma, papillary, familial and sporadic; retinoblastoma; rhabdoid predisposition syndrome, familial; rhabdoid tumors; rhabdomyosarcoma; small-cell cancer of lung; soft tissue sarcoma, squamous cell carcinoma, head and neck; T-cell acute lymphoblastic leukemia; Turcot syndrome with glioblastoma; tylosis with esophageal cancer; uterine cervix carcinoma, Wilms' tumor, type 2; and Wilms' tumor, type 1, etc.

The cancer may be metastatic or non-metastatic.

According to another embodiment, the 1′-2′ gcADPR and 1′-3′ gcADPR may be synthesized in bacterial cells (as further described herein below) and the bacteria may be used as a carrier. In one embodiment, the bacteria are selected as being capable of homing to a tumor—examples of such bacteria are provided in WO2021/205444, the contents of which is incorporated herein by reference.

Since 1′-2′ gcADPR (or a salt, an enantiomer, solvate or hydrate thereof) increases immune response, the present inventors contemplate that 1′-2′ gcADPR and 1′-3′ gcADPR may be administered/co-formulated with an immunomodulatory agent.

Examples of immunomodulatory agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1; and immunomodulatory drugs including, but not limited to lenalidomide (Revlimid).

Exemplary anti-PDI antibodies include Pembrolizumab (Keytruda), Nivolumab (Opdivo), Cemiplimab (Libtayo) and Dostarlimab (Jemperli).

Additional anti-PD1 antibodies include JTX-4014, Spartalizumab (PDR001), Camrelizumab (SHR1210), Sintilimab (IBI308), Tislelizumab (BGB-A317), Toripalimab (JS 001) INCMGA00012 (MGA012), AMP-224 AMP-514 (MEDI0680).

For the treatment of cancer, the 1′-2′ gcADPR and 1′-3′ gcADPR (or a salt, an enantiomer, solvate or hydrate thereof) may be administered/co-formulated with a chemotherapeutic agent.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Since 1′-2′ gcADPR and 1′-3′ gcADPR, each alone or in combination, promote immune activity, the present inventors further contemplate that 1′-2′ gcADPR and 1″-3′ gcADPR may serve as an adjuvant in vaccines.

Thus, according to another aspect of the present invention there is provided a vaccine comprising a disease-associated antigen and an adjuvant comprising 1′-2′ gcADPR, or a salt, an enantiomer, solvate or hydrate thereof.

According to another aspect of the present invention there is provided a vaccine comprising a disease-associated antigen and an adjuvant comprising 1′-3′ gcADPR, or a salt, an enantiomer, solvate or hydrate thereof.

As used herein, the term “vaccine” refers to a pharmaceutical preparation or product that upon administration induces an immune response, e.g. a cellular immune response, which specifically recognizes and attacks a pathogen (or a diseased cell such as a cancer cell). The vaccine of the present invention preferably also includes an immunologically acceptable carrier.

The term “adjuvant” as used herein refers to a substance that increases the ability of an antigen to stimulate the immune system.

The disease associated antigen is typically a protein (or nucleic acid encoding same) or a fragment or fragments thereof which are antigenic-i.e. capable of eliciting an immune response in the subject being immunized. Any length of the fragment is contemplated as long as it is able to elicit the immune response in the subject being vaccinated.

Disease-associated antigens of this aspect of the present invention include but are not limited to cancer antigens, infectious disease antigens such as bacterial antigens, viral antigens, fungal antigens or parasitic antigens, allergy antigens, autoimmune antigens and mixtures of these antigens.

In another embodiment, the disease associated antigen is a short peptide corresponding to one or more antigenic determinants of a protein. The peptide typically binds to a class I or II MHC receptor thus forming a ternary complex that can be recognized by a T-cell bearing a matching T-cell receptor binding to the MHC/peptide complex with appropriate affinity. Peptides binding to MHC class I molecules are typically about 8-14 amino acids in length. T-cell epitopes that bind to MHC class II molecules are typically about 12-30 amino acids in length. In the case of peptides that bind to MHC class II molecules, the same peptide and corresponding T cell epitope may share a common core segment, but differ in the overall length due to flanking sequences of differing lengths upstream of the amino-terminus of the core sequence and downstream of its carboxy terminus, respectively. A T-cell epitope may be classified as an antigen if it elicits an immune response.

In another embodiment, the disease-associated antigen is a neoantigen.

As used herein the term “neoantigen” is an epitope that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

Since 1′-2′ gcADPR is thought to promote the immune response to an agent, agents which down-regulate or sequester 1′-2′ gcADPR can be used to reduce the immune response. Similarly, since 1′-3′ gcADPR is thought to promote the immune response to an agent, agents which down-regulate or sequester 1′-3′ gcADPR can be used to reduce the immune response. This may be particularly important for treating autoimmune diseases which are associated with an enhanced immune response.

Thus, according to another aspect of the present invention there is provided a method of treating an autoimmune disease in a subject in need thereof or a disease associated with neuronal degeneration comprising administering to the subject a therapeutically effective amount of a Tad1 polypeptide having an amino acid sequence at least 80% identical to any one of SEQ ID NOs: 1-11, or a polynucleotide encoding the polypeptide, the polypeptide comprising a 1′-2′ gcADPR and/or 1′-3′ gcADPR sequestering activity, thereby treating the autoimmune disease or the disease associated with neuronal degeneration.

The term “Tad1 polypeptide” refers to a protein having a sequence at least 80%, identical, at least 81% identical, at least 82% identical, at least 83% identical, at least 84% identical, at least 85% identical, at least 86% identical, at least 87% identical at least 88% identical, or at least 89% identical 90%, identical, at least 91% identical, at least 92% identical, at least 93% identical, at least 94% identical, at least 95% identical, at least 96% identical, at least 97% identical at least 98% identical, or at least 99% identical to any one of SEQ ID NOs: 1-11, which is able to sequester 1′-2′ gcADPR and/or 1′-3′ gcADPR.

Percent identity can be determined using any homology comparison software, including for example, the BlastP software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

An exemplary method for ascertaining whether the protein is capable of sequestering 1′-2′ gcADPR is by analyzing the NADase activity of ThsA enzyme in the presence of 1′-2′ gcADPR—see for example Shultz et al., Methods Mol Biol. 2018; 1813:77-90. doi:10.1007/978-1-4939-8588-3_6, the contents of which are incorporated herein by reference.

An exemplary ThsA enzyme is Bacillus cereus MSX-D12 ThsA enzyme (e.g. having an amino acid sequence as set forth in SEQ ID NO: 24). It will be appreciated that ThsA homologs from other bacteria are also contemplated.

Additional information on analyzing NADase activity as a reporter for 1′-2′ gcADPR is provided in the Examples section herein below. A reduction in NADase activity will be observed if the Tad1 polypeptide is capable of sequestering 1′-2′ gcADPR.

As mentioned, the Tad1 polypeptide may be provided as a protein per se or as a polynucleotide agent (i.e. DNA sequence) which encodes the protein.

Tad1 DNA sequences are typically inserted into expression vectors to enable expression of the recombinant polypeptide. The expression vector typically includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

In addition to the elements already described, the expression vector may contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategen, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

A variety of prokaryotic or eukaryotic cells can be used as host-expression systems to express the Tad1 polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by some embodiments of the invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Recombinant viral vectors may also be used to synthesize Tad1 polypeptides of the present invention. Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Exemplary autoimmune diseases include, but are not limited to, rheumatoid arthritis (RA), lupus (SLE), atherosclerosis, multiple sclerosis (MS), hashimoto disease, type I diabetes, autoimmune pancreatitis, graft-versus-host disease (GVHD), sepsis, Ebola, avian influenza, smallpox, systemic inflammatory response syndrome (SIRS), hemophagocytic lymphohistiocytosis, Crohn's and ulcerative colitis, familial Mediterranean fever (FMF), TNF receptor-associated periodic syndrome (TRAPS), hyperimmunoglobulinemia D with periodic fever syndrome (HIDS), familial cold autoinflammatory syndrome (FCAS), the Muckle-Wells syndrome (MWS), neonatal-onset multisystem inflammatory disease (NOMID), deficiency of ADA2 (DADA2), NLRC4 inflammasomopathies, X-linked lymphoproliferative type 2 disorder (XLP), the Takenouchi-Kosaki syndrome, and the Wiskott-Aldrich syndrome (WAS).

As mentioned, the present inventors also contemplate administration of Tad-1 proteins for treatment of neuronal injury. The injury may be brought about by a disease e.g. a neurodegenerative disease, stroke or by an injury per se, such as a traumatic brain injury, a spinal cord injury, a peripheral nerve injury or an eye injury.

Exemplary neurodegenerative diseases which may be treated using 1′-2′ gcADPR (or a salt, an enantiomer, solvate or hydrate thereof) include, but are not limited to Amyotrophic Lateral Sclerosis (ALS), Parkinson's disease, Multiple System Atrophy (MSA), Huntington's disease, Alzheimer's disease, Rett Syndrome and Multiple Sclerosis (MS).

The 1′-2′ gcADPR (or a salt, an enantiomer, solvate or hydrate thereof) or the Tad-1 protein may be used per se or as part of a pharmaceutical composition, where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the 1′-2′ gcADPR (or a salt, an enantiomer, solvate or hydrate thereof) or the Tad1 protein, accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. 1′-2′ gcADPR) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

The present inventors further contemplate using ThsA enzymes in order to screen for agents that regulate immune modulation.

Thus, according to still another aspect of the present invention there is provided a method of screening for an agent that modulates immune modulation comprising:
contacting the agent with a ThsA enzyme;
measuring the NADase activity of the ThsA enzyme wherein a change in NADase activity is indicative of an agent that modulates immune modulation.

In one embodiment, the agent increases immune modulation-such agents increase the NADase activity of ThsA.

In another embodiment, the agent decreases immune modulation-such agents decrease the NADase activity of ThsA.

ThsA enzymes and methods of measuring NADase activity have been described herein above.

In one embodiment, the amount of NADase activity is compared to the amount of NADase activity when the same assay is carried out in the presence of 1′-2′ gcADPR.

In another embodiment, the contacting may be carried out in the presence of a Tad1 polypeptide.

In another embodiment, the contacting may be carried out in the presence of a 1′-2′ gcADPR.

The present invention contemplates screening any agent for immune modulating activity including small molecule agents and 1′-2′ gcADPR derivatives.

Once an agent has been identified as having immune modulating activity, it's therapeutic activity may be tested in animal models of immune diseases.

An exemplary method of producing and isolating 1′-2′ gcADPR is provided herein below:
1. co-express a TIR domain protein and Tad1 protein in bacterial cells (e.g. E. coli cells)—this promotes the synthesis of 1′-2′ gcADPR by the TIR domain protein and the formation of a complex between Tad1 protein and 1′-2′ gcADPR;
2. prepare cell lysates from the bacterial cells; and
3. isolate the complex.

Another exemplary method of producing and isolating 1′-2′ gcADPR is as follows:
1. infect bacterial cells (e.g. E. coli or B. subtilis) that express ThsB (e.g. as set forth in SEQ ID NO: 25) with bacteriophages which do not express Tad1 and that are sensitive to the Thoeris system. Examples include phages SBSphiJ, SBSphiJ1, SBSphi J2.
2. generating a cell lysate of the infected bacterial cells before the phage lyses the cells;
3. contacting the cell lysate with Tad1 to generate a complex between Tad1 protein and 1′-2′ gcADPR.
4. isolate the complex.

In one embodiment, the Tad1 protein is expressed with a separating moiety (i.e. affinity tag) such that it is possible to isolate the complex using an affinity chromatography technique.

Examples of separating moieties include but are not limited to polyhistidine tags, polyarginine tags, glutathione-S-transferase, biotin, maltose binding protein, S-tag, influenza virus HA tag, thioredoxin, staphylococcal protein A tag, the FLAG™ epitope, AviTag epitope, and the c-myc epitope.

Alternatively, the complex can be isolated directly using an antibody that binds specifically to Tad1.

Examples of TIR domain proteins that can be used to isolate 1′-2′ gcADPR include plant Brachypodium distachyon (BdTIR-having an amino acid sequence as set forth in SEQ ID NO: 26), which was shown to constitutively produce the cADPR isomer molecule when expressed in E. coli (Wan, L. et al. TIR domains of plant immune receptors are NAD+-cleaving enzymes that promote cell death. Science 365, 799-803 (2019)). Additional contemplated TIR domains which may be expressed include the TIR domains from drosophil SARM1 (SEQ ID NO: 27) or human SARM1 (SEQ ID NO: 12), and homologs thereof.

Following isolation of the complex, 1′-2′ gcADPR may be purified from Tad1 by denaturing the protein (e.g. by heating the complex-for example to a temperature above 85° C. for at least 5 minutes or denaturing agents known in the art such as proteases and ethanol. 1′-2′ gcADPR may be further purified using methods known in the art including but not limited to filtration, size exclusion chromatography, high performance liquid chromatography and reversed-phase chromatography.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Phage strains, isolation, cultivation and sequencing: Phage SBSphiJ was isolated as described in Doron, S. et al. Science 359, eaar4120 (2018). Other phages were isolated from soil samples on B. subtilis BEST7003 culture as described in Doron et al infra. For this, soil samples were added to a log phase B. subtilis BEST7003 culture and incubated overnight to enrich for B. subtilis phages. The enriched sample was centrifuged and filtered through 0.45 μm filters, and the filtered supernatant was used to perform double layer plaque assays as described in Kropinski et al.²³. Single plaques that appeared after overnight incubation were picked, re-isolated 3×, and amplified as described below.

Phages were propagated by picking a single phage plaque into a liquid culture of B. subtilis BEST7003 grown at 37° C. to OD₆₀₀ of 0.3 in magnesium manganese broth (MMB) (LB+0.1 mM MnCl₂+5 mM MgCl₂) until culture collapse. The culture was then centrifuged for 10 minutes at 3,200×g and the supernatant was filtered through a 0.2 μm filter to get rid of remaining bacteria and bacterial debris.

High titer phage lysates (>10⁷ pfu/ml) were used for DNA extraction. 500 μl of the phage lysate was treated with DNase-I (Merck cat #11284932001) added to a final concentration of 20 mg mL⁻¹ and incubated at 37° C. for 1 h to remove bacterial DNA. DNA was extracted using the QIAGEN DNeasy blood and tissue kit (cat #69504) starting from the Proteinase-K treatment step to lyse the phages. Libraries were prepared for Illumina sequencing using a modified Nextera protocol as previously described²⁴.

Following Illumina sequencing, adapter sequences were removed from the reads using Cutadapt version 2.8²⁵ with the option −q 5. The trimmed reads from each phage genome were assembled into scaffolds using SPAdes genome assembler version 3.14.0²⁶, using the-careful flag. Each assembled genome was analyzed with Prodigal version 2.6.3²⁷ (default parameters) to predict ORFs.

Plaque assays: Phage titer was determined using the small drop plaque assay method²⁸. 400 μL of the bacterial culture was mixed with 30 mL melted MMB 0.5% agar, poured on 10 cm square plates, and let to dry for 1 h at room temperature. In cases of bacteria expressing anti-defense candidates, 1 mM IPTG was added to the medium. In cases of bacteria expressing dCas9-gRNA constructs, 0.002% xylose was added to the medium. 10-fold serial dilutions in MMB were performed for each of the tested phages and 10 μl drops were put on the bacterial layer. After the drops had dried up, the plates were inverted and incubated at room temperature overnight. Plaque forming units (PFUs) were determined by counting the derived plaques after overnight incubation and lysate titer was determined by calculating PFUs per ml. When no individual plaques could be identified, a faint lysis zone across the drop area was considered to be 10 plaques. Efficiency of plating (EOP) was measured by comparing plaque assay results on control bacteria and bacteria containing the defense system and/or a candidate anti-defense gene.

Pulldown attempts of ThsA and ThsB with tagged Tad1: Bacillus subtilis phage SBSphiJ7 tad1 gene was cloned into the pBbA6c plasmid containing a C-terminal 8× His-tag and a lac promoter³⁵. The Bacillus cereus MSX-D12 Thoeris thsA and thsB genes were cloned as an operon under the arabinose-inducible promoter on pBAD. Both plasmids were co-transformed into an E. coli MG1655 strain.

Overnight cultures containing the pBAD-thsA-thsB and pBbA6c-tad1-His constructs were diluted 1:100 in 100 mL MMB and grown at 25° C., 200 rpm shaking. 1 mM IPTG and 0.2% arabinose were added at an OD₆₀₀ of 0.3 and harvested after 3 h by centrifugation at 4000 RPM for 20 min at 4° C. Pellets were stored at −80° C. and then lysed with 250 μL 50 mM Na-Phosphate buffer pH 8.0, 0.1 M NaCl and 1 mg mL⁻¹ lysozyme (Sigma cat #L6876). Pellets resuspended with lysozyme were shaken for 10 min at 25° C. and then 750 μL of NTA-washing buffer (Na-Phosphate buffer 20 mM pH 7.4, 0.5 M NaCl, 20 mM imidazole, 0.05% Tween 20) was added. The samples were transferred to a FastPrep Lysing Matrix B in a 2 ml tube (MP Biomedicals cat #116911100) and lysed using FastPrep bead beater for 2×40 s at 6 m s⁻¹. Tubes were then centrifuged at 4° C. for 10 min at 15,000×g.

The supernatant was then applied to Ni-NTA-magnetic beads (Qiagen 36113) and washed twice with 0.5 mL NTA-washing buffer, followed by elution with NTA-washing buffer containing 500 mM imidazole. Protein samples containing Tad1-His co-expressed with ThsA and ThsB were compared to cells expressing either Tad1-His or ThsA/ThsB by running the purified samples on a Bolt™ 4 to 12%, Bis-Tris protein gel (Thermofisher NW04122BOX).

Preparation of filtered cell lysates: For the in vivo experiments, Bacillus subtilis BEST7003 co-expressing a Thoeris system was used in which ThsA was mutated in its NADase active site (ThsA_{N112A}+ThsB)¹ under a native promoter, and Tad1 under the Physpank promoter. Controls included cells expressing only the mutated Theoris system, as well as cells lacking both the Theoris system and Tad1. These cultures were grown overnight and then diluted 1:100 in 350 mL MMB supplemented with 1 mM IPTG and grown at 37° C., 200 rpm shaking for 90 min. Each culture was then incubated and shaken at 25° C. 200 rpm until reaching an OD₆₀₀ of 0.3. At this point, a sample of 50 mL was taken as the uninfected (time 0 min) sample, and SBSphiJ phage was added to the remaining 300 mL culture at an MOI of 5. Flasks were incubated at 25° C. with shaking (200 rpm), for the duration of the experiment. 50 ml samples were collected at time points 75, 90, 105, and 120 min post-infection. Immediately upon sample removal (including time point 0 min), the 50 mL sample tubes were placed on ice and centrifuged at 4° C. for 10 min to pellet the cells. The supernatant was discarded and the tube was flush frozen and stored at −80° C.

For the in vitro experiment with purified cmTad1, Bacillus subtilis BEST7003 cultures overexpressing the Theoris ThsB protein under the Physpank promoter, together with control cultures, were diluted 1:100 in 200 mL MMB supplemented with 0.1 mM IPTG and grown at 37° C., with shaking at 200 rpm. After 90 min, temperature was lowered to 25° C. and shaken (200 rpm) until reaching an OD₆₀₀ of 0.3. Then, SBSphiJ phage was added to the culture at an MOI of 5. Flasks were incubated at 25° C. with shaking (200 rpm). 120 min post infection the culture was collected into 50 ml tubes, centrifuged for 10 min at 4° C., supernatant discarded and pellet was stored at −80° C.

To extract the cell metabolites from frozen pellets, 600 μl of 100 mM phosphate buffer, pH 8.0, supplemented with 4 mg mL⁻¹ lysozyme (Sigma cat #L6876) was added to each pellet. Tubes were then incubated for 10 min at 25° C., and returned to ice. The samples were transferred to a FastPrep Lysing Matrix B in a 2 mL tube (MP Biomedicals cat #116911100) and lysed using FastPrep bead beater for 2×40 s at 6 m s⁻¹. Tubes were then centrifuged at 4° C. for 10 min at 15,000×g. Supernatant was transferred to Amicon Ultra-0.5 Centrifugal Filter Unit 3 kDa (Merck Millipore cat #UFC500396) and centrifuged for 45 min at 4° C., 12,000×g. Filtered lysates were taken either for LC-MS analysis or for in vitro NADase activity assays.

LC-MC monitoring of the Thoeris cADPR isomer: Sample analysis was carried out by MS-Omics as follows. Samples were diluted 1:3 in 10% ultra-pure water and 90% acetonitrile containing 10 mM ammonium acetate at pH 9 then filtered through a Costar Spin-X centrifuge tube filter 0.22 μm Nylon membrane. The analysis was carried out using a UPLC system (Vanquish, Thermo Fisher Scientific) coupled with a high-resolution quadrupole-orbitrap mass spectrometer (Q Exactive™ HF Hybrid Quadrupole-Orbitrap, Thermo Fisher Scientific). The standard cADPR peak was identified using a synthetic standard (cADPR: Sigma-Aldrich, C7344) run. The UPLC was performed using an Infinity Lab PoroShell 120 hydrophilic interaction chromatography (HILIC-Z PEEK) lined column with dimensions of 2.1×150 mm and a particle size of 2.7 μm (Agilent Technologies). The composition of mobile phase A was 10 mM ammonium acetate at pH 9 in 90% acetonitrile LC-MC grade (VWR Chemicals) and 10% ultra-pure water from Direct-Q 3 UV Water Purification System with LC-Pak Polisher (Merck KGaA) and mobile phase B was 10 mM ammonium acetate at pH 9 in ultra-pure water with 15 μM medronic acid (InfinityLab Deactivator additive, Agilent Technologies). The flow rate was kept at 250 μL mL⁻¹ consisting of a 2 min hold at 10% B, increased to 40% B at 14 min, held till 15 min, decreased to 10% B at 16 min and held for 8 min. The column temperature was set at 30° C. and an injection volume of 5 μL. Analysis was performed in positive ionization mode from m/z 200 to 1000 at a mass resolution of 120,000 (at m/z 200). An electrospray ionization interface was used as ionization source. Peak areas were extracted using TraceFinder 4.1 (ThermoFisher Scientific) with an accepted deviation of 5 ppm. Fragmentation was done through a higher-energy collisional dissociation cell using a normalized collision energy of 20, 40 and 60 eV where the spectrum is the sum of each collision energy.

NADase activity assay as a reporter for the cADPR isomer: NADase assay was performed by using the Bacillus cereus MSX-D12 ThsA enzyme as a reporter for the presence of the cyclic ADPR isomer¹. The reporter enzyme ThsA was expressed and purified as described previously¹. NADase reaction was performed in black 96-well half area plates (Corning cat #3694) at 25° C. in a 50 μl final reaction volume. 5 μL of 5 mM nicotinamide 1,N6-ethenoadenine dinucleotide (εNAD, Sigma, N2630) solution was added to each well sample immediately prior to measurements and mixed by pipetting. εNAD was used as a fluorogenic substrate to report the ThsA enzyme NADase activity by monitoring increase in fluorescence (excitation 300 nm/emission 410 nm) using a Tecan Infinite M200 plate reader at 25° C. Reaction rate was derived from the linear part of the initial reaction.

For the assessment of the in vivo activity of Tad1 in cells co-expressing both ThsB (native promoter) and Tad1, filtered lysates were mixed directly with ThsA, followed by the addition of εNAD. Controls included filtered lysates derived from cells expressing ThsB (without Tad1), and cells expressing neither ThsB nor Tad1. For the assessment of the in vitro activity of purified cmTad1, phage-infected cell filtered lysate (diluted 1:16-1:20) overexpressing ThsB (100 μM IPTG) were incubated with purified cmTad1 (150-600 nM final concertation) at 25° C. Controls included filtered lysates incubated with diluted SEC buffer.

To examine the level of the cADPR isomer after cmTad1 heat-inactivation, cmTad1 (600 nM) was incubated with phage-infected cell filtered lysate (diluted 1:16) for 10 min at 25° C. followed by incubation at either 85° C. or 25° C. for an additional 5 min. Samples were then mixed with 2 μL of 2.5 μM ThsA and 5 μl of 5 mM εNAD and fluorescence was monitored as descried above.

Tad1 crystallization and structural analysis: For crystallography experiments, cmTad1 and cbTad1 genes were cloned from synthetic DNA fragments (Integrated DNA Technologies) by Gibson assembly into a custom pET expression vector containing an N-terminal 6× His-SUMO2 tag as previously described³⁹. Plasmids were transformed into BL21(DE3) RIL E. coli (Agilent) and colonies were grown on MDG agar plates (1.5% agar, 2 mM MgSO₄, 0.5% glucose, 25 mM Na₂HPO₄, 25 mM KH₂PO₄, 50 mM NH₄Cl, 5 mM Na₂SO₄, 0.25% aspartic acid, and 2-50 μM trace metals). Three colonies were picked into a 30 mL MDG starter culture and grown overnight at 37° C. with 230 rpm shaking. 15 ml of overnight cultures were used to seed 1 L M9ZB expression cultures (2 mM MgSO₄, 0.5% glycerol, 47.8 mM Na₂HPO₄, 22 mM KH₂PO₄, 18.7 mM NH₄Cl, 85.6 mM NaCl, 1% Cas-Amino acids, 2-50 μM trace metals, 100 μg mL⁻¹ ampicillin, and 34 μg mL⁻¹ chloramphenicol). Expression cultures were grown at 37° C. with 230 rpm shaking to an OD₆₀₀ of 2-2.5 before inducing expression with 0.5 mM IPTG and incubating cultures at 16° C. with 230 rpm shaking for 16-20 h. Selenomethionine-labeled protein was produced as previously described by growing expression cultures in modified M9ZB media (2 mM MgSO₄, 0.4% glucose, 47.8 mM Na₂HPO₄, 22 mM KH₂PO₄, 18.7 mM NH₄Cl, 85.6 mM NaCl, 2-50 UM trace metals, 1 μg mL⁻¹ thiamine, 100 μg mL⁻¹ ampicillin, 34 μg mL⁻¹ chloramphenicol)⁴⁰. After expression, 2 L of culture was harvested by centrifugation and resuspended in 120 mL lysis buffer (20 mM HEPES-KOH pH 7.5, 400 mM NaCl, 30 mM imidazole, 10% glycerol, 1 mM DTT). Cells were lysed by sonication then clarified by centrifugation at 25,000×g for 20 min, and lysate was passed over 8 mL Ni-NTA resin (Qiagen) using gravity chromatography. Resin was washed with 70 mL wash buffer (20 mM HEPES-KOH pH 7.5, 1 M NaCl, 30 mM imidazole, 10% glycerol, 1 mM DTT) and 20 mL lysis buffer, then eluted with lysis buffer containing 300 mM imidazole. Eluate was dialyzed overnight using 14 kDa dialysis tubing in dialysis buffer (20 mM HEPES-KOH pH 7.5, 250 mM KCl, 1 mM TCEP) in the presence of recombinant human-SENP2 to induce SUMO-tag cleavage, before further purification by size-exclusion chromatography using a Superdex 75 16/600 column (Cytiva). Size-exclusion peaks of interest were collected and concentrated to >40 mg mL⁻¹, then flash frozen and stored at −80° C.

Ligand-bound Tad1 was produced by first co-expressing Tad1 with BdTIR. BdTIR was cloned into a custom pET vector containing a C-terminal Twin-Strep tag, a chloramphenicol resistance gene, and IPTG-inducible promoter. Plasmids containing cmTad1 or cbTad1 were co-transformed with BdTIR into BL21(DE3) cells, then plated onto MDG agar plates and expressed as described above. Ligand-bound Tad1 was purified as described above with a modified method. After elution from NiNTA resin using 300 mM imidazole, eluate was treated with recombinant human-SENP2 for 1 h to induce SUMO-tag cleavage then immediately purified further by size-exclusion chromatography. Peaks of interest were collected and concentrated to >25 mg mL⁻¹, then flash frozen and stored at −80° C. To purify 1′-2′ gcADPR from Tad1, cmTad1 was boiled for 5 min at 95° C., centrifuged for 10 min at 17,000×g, and filtered through a 10 kDa concentration unit (Amicon). Filtrate was collected, diluted in water, and stored at −20° C.

Crystals of cbTad1 were grown using the hanging drop method using EasyXtal 15-well trays (NeXtal). Sample was prepared by first diluting purified protein to 10 mg mL⁻¹ using buffer containing 20 mM HEPES-KOH pH 7.5, 80 mM KCl, and 1 mM TCEP. 2 μL hanging drops were set at a 1:1 ratio of protein to reservoir solution over a well with 400 μL reservoir solution. Each protein was crystallized using the following conditions: 1) Native or selenomethionine-labeled cbTad1 in the apo state: Crystals were grown for 1-2 weeks using reservoir solution containing 0.1 M Tris-HCl pH 7.5 and 40% PEG 200 before being harvested by flash freezing in liquid nitrogen. 2) cbTad1 complexed with 1′-2′ gcADPR: Using cbTad1 purified from cells expressing BdTIR, crystals were grown for 1-5 days using reservoir solution containing 0.1 M MES pH 5.0, 1% PEG-6000, and 300 M 1′-2′ gcADPR before being cryo-protected with reservoir solution containing 35% ethylene glycol and harvested by flash freezing in liquid nitrogen. X-ray diffraction data were collected at the Advanced Photon Source (beamlines 24-ID-C and 24-ID-E), and data were processed using the SSRL autoxds script (A. Gonzalez, Stanford SSRL). Anomalous data for phase determination were collecting using selenomethionine-labeled crystals. Heavy sites were identified, and initial maps were produced using AutoSol in Phenix⁴¹. Model building was performed in Coot⁴², with refinement in Phenix and statistics were analyzed. Final structures were refined to stereochemistry statistics for Ramachandran plot (favored/allowed), rotamer outliers, and MolProbity score as follows: cbTad1 apo, 98.60%/1.40%, 0.20%, 1.11; cbTad1-1′-2′ gcADPR, 97.97%/2.03%, 0.91%, 1.09.

Purification and validation of 1′-2′-gcADPR: To purify 1′-2′ gcADPR, concentrated (>1 mM) ligand-bound cmTad1 in storage buffer (20 mM HEPES-KOH pH 7.5, 250 mM KCl, 1 mM TCEP) was denatured by boiling at 95° C. for 15 min. Denatured cmTad1 was pelleted at 13,500×g for 20 min, and supernatant was passed through a 0.5 mL⁻¹⁰ kDa filter (Amicon). 1′-2′ gcADPR concentration was estimated using the extinction coefficient of cADPR and absorbance at 260 nm, which was measured using a Denovix Ds-11+spectrophotometer.

To validate the signaling capability of 1′-2′ gcADPR isolated from ligand-bound cmTad1, 200 μM ligand-bound cmTad1 was subjected to temperatures of either 25° C. or 95° C. for 15 min before centrifugation and 10 kDa filtration, and ThsA activity of the resulting filtrate was measured through &NAD fluorescence as described above.

High-performance liquid chromatography (HPLC) was used to further validate the identity of 1′-2′ gcADPR isolated from ligand-bound cmTad1. 500 μM filtrate was compared to similar amounts of cADPR and ADPR diluted in storage buffer. Individual samples were injected onto a C18 column (Zorbax Bonus-RP 4.6×150 mm, 3.5 μm) attached to an Agilent 1200 Infinity Series LC system and eluted isocratically at 40° C. with a flow rate of 1 mL min 1 with 50 mM Na₂HPO₄ pH 6.8 supplemented with 3% acetonitrile. Elution profiles were monitored at an absorbance of 254 nm.

SARM1 lysate experiments: 200 mL MMB 1:100 was inoculated with 2 ml of overnight cultures expressing a TIR-domain-containing subsequence of human SARM1 or drosophila SARM1. Culture was shaken (210 RPM) for 1.5 hr in 37° C., then temperature was lowered to 30° C. When culture reached an OD of 0.5 (3 hr from time of inoculation), 0.2 mM IPTG was added to culture. Culture was shaken at 210 RPM for 3.5 hr until harvested by centrifugation (3900 RPM) for 10 min at 4° C. with each 50ml culture centrifuged in a separate tube. The supernatant was discarded and the pellets flesh-freezed at −80° C. The pellets were kept at −80° C. Pellets were then lysed and assayed with ThsA NADase activity assay as described above.

### Results

A group of closely-related phages (including eight phages similar to phage SBSphiJ, a lytic Myoviridae phage with an ˜150 kb-long genome) that infect Bacillus subtilis was isolated. Despite the high sequence similarity between these phages, each phage had 4-5 genes that were not found in the genomes of other phages in the group. (FIG. 1A). The Thoeris defense system protected against all phages from the SBSphiJ group except for phage SBSphiJ7 (FIG. 1B). It was therefore hypothesized that one or more genes that are unique to SBSphiJ7 allow this phage to escape or inhibit the activity of Thoeris.

To test this hypothesis, five genes that were unique to phage SBSphiJ7 were expressed under the control of an inducible promoter, in B. subtilis cells that also express the Thoeris system from Bacillus cereus MSX-D12. One of these genes, which is denoted tad1 (Thoeris anti-defense 1) robustly inhibited the activity of Thoeris, as phages that were normally blocked by Thoeris were able to infect Thoeris-expressing cells if these cells also expressed Tad 1 (FIG. 1B, 1C). Silencing the expression of Tad1 in SBSphiJ7 using dCas9⁹ caused SBSphiJ7 to be blocked by Thoeris, verifying that tad1 is the gene responsible for the Thoeris-inhibiting phenotype of SBSphiJ7 (FIG. 1D).

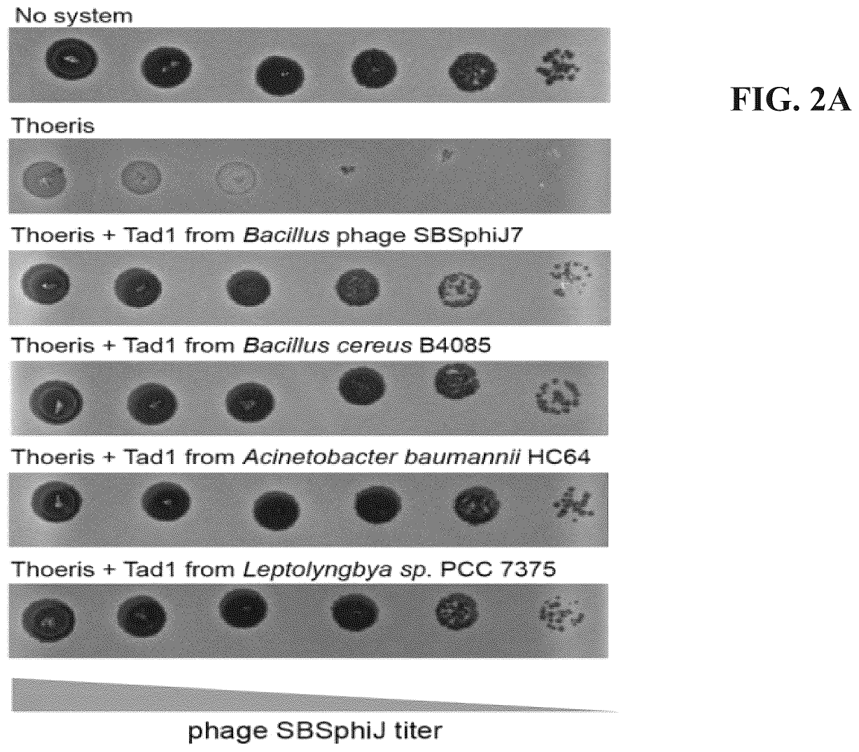

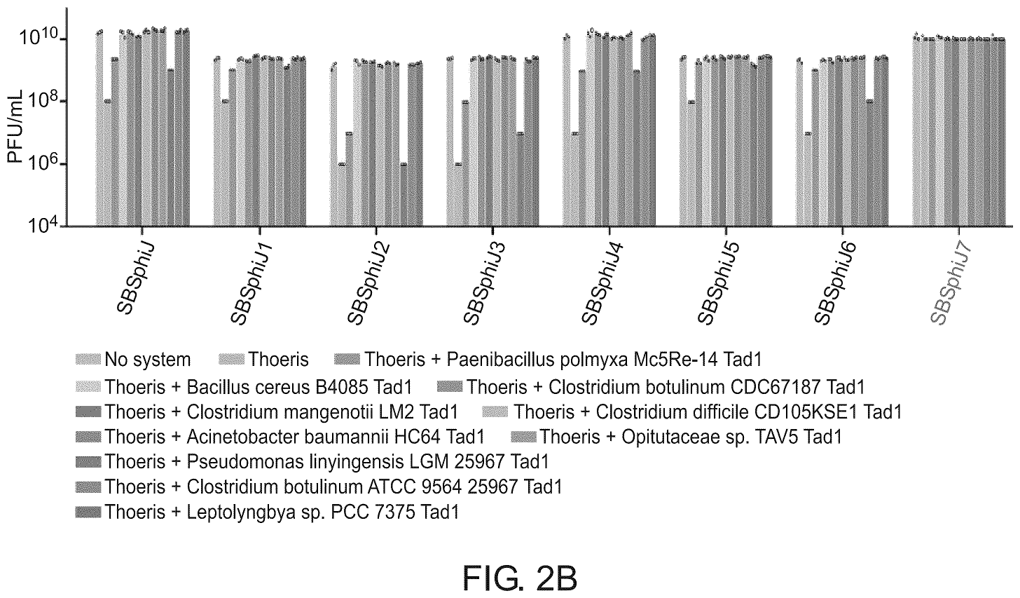

Ten Tad1 homologs that span the phylogenetic diversity of the Tad1 family were into B. subtilis cells that express the Thoeris system. All 10 Tad1 family proteins were able to inhibit Thoeris, including homologs derived from phages that infect distant organisms such as Leptolyngbya sp., Opitutaceae sp. and Acinetobacter baumannii (FIGS. 2A-B). Together, these results reveal a large family of proteins utilized by phages to inhibit the activity of the Thoeris bacterial defense system.

The hallmark of Thoeris defense are TIR-domain ThsB proteins which, once they sense the infection, produce a signaling molecule that triggers the NADase activity of the Thoeris ThsA protein¹. To test if this signaling molecule is produced in the presence of Tad1, cells were engineered to express a Thoeris system in which ThsA was mutated in its NADase active site such that only ThsB is active. These cells were infected with phage SBSphiJ that naturally lacks Tad1, the cells were lysed and the lysates filtered to enrich for molecules smaller than 3 kDa (FIG. 3A). As expected, purified ThsA protein incubated with these filtered lysates in vitro showed strong NADase activity, indicating that the TIR-domain ThsB protein produced the signaling molecule within the cell in response to SBSphiJ infection (FIG. 3B). However, filtered lysates derived from cells in which Tad1 was co-expressed with the active ThsB failed to activate ThsA in vitro, suggesting that the signaling molecule was eliminated or inactivated in Thoeris-infected cells that co-express Tad1 (FIG. 3B). Liquid chromatography followed by mass spectrometry (LC-MS) confirmed that the signaling molecule, having a mass identical to the mass of cADPR, was present in lysates of infected cells expressing the TIR-domain protein ThsB, but absent in lysates derived from infected cells where ThsB was co-expressed with Tad1 (FIG. 3C). These results indicate that Tad1 does not inhibit the Thoeris effector protein ThsA, but rather inhibits Thoeris upstream of the ThsA protein.

Next, a purified Tad1 homolog from a prophage integrated in Clostridioides mangenotii (cmTad1) was tested to see if it can directly eliminate the signaling molecule from the lysate. Filtered lysates were collected from cells overexpressing ThsB that were infected by phage SBSphiJ. The lysates were incubated with cmTad1 for 10 minutes (FIG. 3D). Lysates incubated with purified cmTad1 completely lost the ability to induce the NADase activity of ThsA, demonstrating that Tad1 rapidly eliminates the signaling molecule from the filtered lysate rather than inhibiting its production (FIG. 3E).

Some phages were previously shown to inhibit bacterial immune signaling, for example cyclic oligoadenylate signaling in type III CRISPR-Cas systems, by introducing enzymes that cleave the signaling molecules¹⁶. It was therefore hypothesized that Tad1 is an enzyme that cleaves the cADPR isomer immune signaling molecule of Thoeris. Under this hypothesis, one would expect Tad1 to deplete the signaling molecule in a time-dependent manner. However, counter to this hypothesis, incubation of sub-inhibitory concentrations of cmTad1 with the filtered lysate for prolonged time did not result in time-dependent increased depletion of the active molecule from the lysate (FIG. 3F). These results implied that Tad1 is not an enzyme, but rather a chelator (a “sponge”) that binds and sequesters the signaling cADPR isomer molecule.

To further examine the hypothesis that Tad1 chelates the cADPR isomer signaling molecule, the present inventors tested whether, following the chelation, cmTad1 denaturation could release the bound signaling molecule into the buffer. For this, cmTad1 was incubated with the signal-containing lysate for 10 minutes, and then denatured by exposure to 85° C. for 5 minutes. It was found that following Tad1 denaturation, the buffer regained the capacity to activate ThsA in vitro (FIG. 3G). These results demonstrate that Tad1 binds and sequesters, but does not degrade, the Thoeris signaling molecule, and that denaturation of Tad1 releases the chelated molecule intact. These results therefore show that Tad1 inhibits Thoeris defense by physically binding and sequestering the ThsB-derived signaling molecule, thus preventing the activation of the Theoris immune effector and mitigating Theoris-mediated defense.

It was previously shown that cADPR isomer molecules produced by plant TIR proteins can activate the Theoris effector ThsA1. The present inventors therefore co-expressed cmTad1 with a TIR-domain protein from the plant Brachypodium distachyon (BdTIR), which was shown to constitutively produce the cADPR isomer molecule when expressed in E. coli t. cmTad1 purified from BdTIR-expressing cells showed substantial shifts during size-exclusion chromatography and exhibited increased absorption at UV₂₆₀ as compared to cmTad1 purified from control cells, suggesting that cmTad1 binds the signaling molecule produced by the plant TIR. Indeed, supernatant collected from heat-denaturation of the molecule-bound cmTad1 was able to activate ThsA, verifying that the signaling molecule produced by the plant TIR, which was bound by cmTad1, is the same molecule utilized by the Theoris system.

Crystal structure of Tad1 reveals the immune signal 1′-2′ glycocyclic ADPR (gcADPR): To define the molecular mechanism of Tad1 anti-Thoeris evasion, the crystal structures of Tad1 from a Clostridium botulinum prophage (cbTad1) in the apo and ligand-bound states were determined. The structure of Tad1 reveals a homodimeric complex with two protomers arranged head-to-tail (FIG. 4A). Each protomer forms an N-terminal anti-parallel β-sheet (β1-β4) and two long C-terminal helices (α1 and α2) that create a wedge-shaped architecture and allow Tad1ₐ and Tad1_{b} to tightly interlock into a compact assembly (FIG. 4A,B). The N-terminal β-sheets join through a β4ₐ-β4_{b} hydrophilic seam to form the front face of the Tad1 assembly, while the C-terminal helices align to create a four-helix bundle that seals the back face (FIG. 4A,B). The tightly locked assembly creates two recessed ligand binding pockets at the top and bottom ends of the Tad1 complex that are each surrounded by four highly-conserved loops within β2-β3, β4-α1, and the C-terminal tail of Tad1ₐ along with α1-α2 donated by the partner protomer Tad1_{b} (FIG. 4B).

Upon ligand-binding, the Tad1 complex undergoes a 3° rotation to close and envelope the TIR-derived signaling molecule. Tad1 loop β4-α1 moves >3.5 Å and the C-terminal residues 116-122 form an ordered lid that together seal the ligand-binding site (FIG. 4C). Exceptionally clear density within the 1.9 Å ligand-bound cbTad1 complex allowed for unambiguous assignment of each atomic position within the TIR-derived signaling molecule, revealing the compound 1′-2′ glycosyl cyclic adenosine diphosphate ribose (1′-2′ gcADPR) (FIG. 4D). In contrast to the canonical cADPR, in which cyclization occurs through the ribose and the adenine nucleobase NI position, these data demonstrate that the signaling molecule produced by bacterial and plant TIR proteins is unexpectedly cyclized through a unique ribose-ribose glycosidic bond. The Tad1 ligand-binding pocket intimately embraces 1′-2′ gcADPR with 11 residues forming base-and linkage-specific contacts (FIG. 4E,F). The 1′-2′ gcADPR adenine base is stacked between cbTad1 F82 and R109, with N92 making sequence-specific contacts to the Hoogsteen edge (FIG. 4E). The diphosphate backbone is bound by three cbTad1 sidechains R57, R109, and R113, and additional peptide-backbone contacts from R57 and G120. Finally, cbTad1 F87 buttresses the adenosine ribose and residues H29, G55 (NH), R78, and N122 coordinate each free OH in the ribose-ribose linkage, explaining the intimate specificity of Tad1 for the unique linkage in 1′-2′ gcADPR (FIG. 4F). Complete enclosure within the ligand-binding pocket explains how Tad1 efficiently sequesters the TIR-derived signal to inactivate Thoeris defense.

A subsequence including the TIR domain of the human and drosophila TIR-domain comprising protein, SARM1, was overexpressed in bacterial cells. The cells were lysed and the lysates were filtered. Both filtered lysates activated ThsA in vitro (FIG. 5), suggesting that the human and drosophila SARM1 proteins produce gcADPR.

### Example 2

An alternative production of gcADPR based on expression of proteins containing TIR domains was performed as detailed below.

(a) Expression of AaTIR and AbTIR in E. coli

Residues 1-144 of a TIR-domain containing protein from Aquimarina amphilecti (NCBI Accession: WP_091411838.1, called “AaTIR”; SEQ ID NO: 28), and residues 157-292 of a TIR-domain containing protein from Acinetobacter baumannii (NCBI Accession: WP_234622687.1, called “AbTIR”; SEQ ID NO: 29) were cloned from synthetic gene fragments into a custom pET-based expression vector with an N-terminal His-SUMO tag and IPTG-inducible T7 promoter using Gibson assembly (NEB). The resulting plasmids were transformed into BL21-DE3-RIL cells (Agilent) and plated onto MDG agar plates (1.5% Bacto agar, 0.5% glucose, 25 mM Na₂HPO₄, 25 mM KH₂PO₄, 50 mM NH₄Cl, 5 mM Na₂SO₄, 0.25% aspartic acid, 50 μM trace metals, 100 μg mL⁻¹ ampicillin, 34 μg ml 1 chloramphenicol). Plates were incubated at 37° C. overnight, and three (3) colonies were used to inoculate 30 mL of liquid MDG broth. MDG broth cultures were grown at 37° C. overnight with shaking (230 RPM) and 10 mL was transferred to 1 L of M9ZB (47.8 mM Na₂HPO₄, 22 mM KH₂PO₄, 18.7 mM NH₄Cl, 85.6 mM NaCl, 1% casamino acids, 0.5% glycerol, 2 mM MgSO₄, 50 μM trace metals, 100 μg mL⁻¹ ampicillin, 34 μg mL⁻¹ chloramphenicol) media. The M9ZB cultures were grown for 5-6 hours until OD₆₀₀=1.5-2.0 when isopropyl-β-D-thiogalactoside was added to a final concentration of 0.5 mM, and the cultures were incubated at 16° C. for ˜16 h. Cultures were harvested by centrifugation at 3,200×g at 4° C. and cell pellets were flash frozen in liquid nitrogen until protein purification.

### (b) Purification

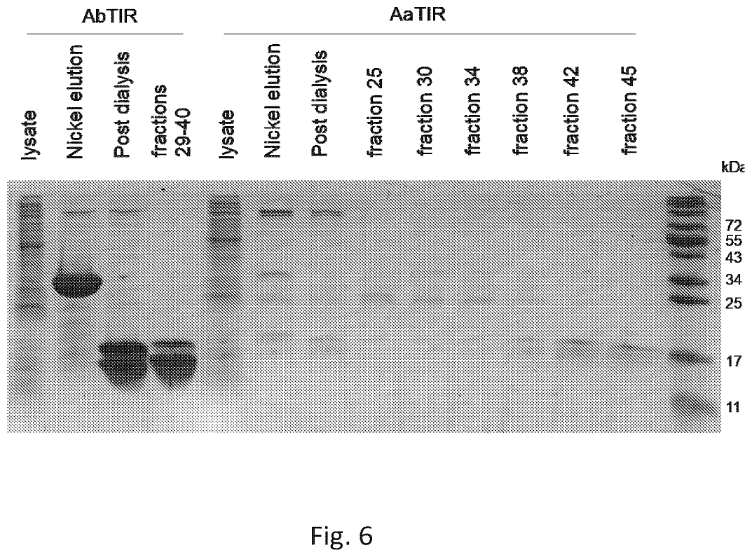

Cell pellets were resuspended in 60 mL of lysis buffer (20 mM HEPES-KOH pH 7.5, 400 mM NaCl, 10% glycerol, 30 mM imidazole, 1 mM TCEP). Lysate was clarified by centrifugation at 50,000×g for 30 min, supernatant was poured over 8 ml Ni-NTA resin (Qiagen), resin was washed with 35 ml lysis buffer supplemented with 1 M NaCl, and protein was eluted with 20 ml lysis buffer supplemented with 300 mM imidazole. Recombinant human SENP2 protease (250 μg) was added, and samples were dialysed overnight at 4° C. in dialysis buffer (10% glycerol, 20 mM HEPES-KOH pH 7.5, 250 mM KCl, 1 mM TCEP), and then purified further by size-exclusion chromatography using a 16/600 Superdex 75 column (Cytiva). Fractions collected from the size exclusion column were analyzed by SDS-PAGE, and fractions showing a band at the expected MW (AaTIR: 17.09 kDa; AbTIR: 15.73 kDa) were collected (FIG. 6) and concentrated to a final concentration of 0.28 mg mL⁻¹ for AaTIR, and 29.8 mg mL⁻¹ for AbTIR using 10-kDa MWCO centrifugal filter units (Millipore Sigma). Samples were then aliquoted, flash frozen in liquid nitrogen and stored at −80° C. until further use.

(c) Synthesis of gcADPR Molecules

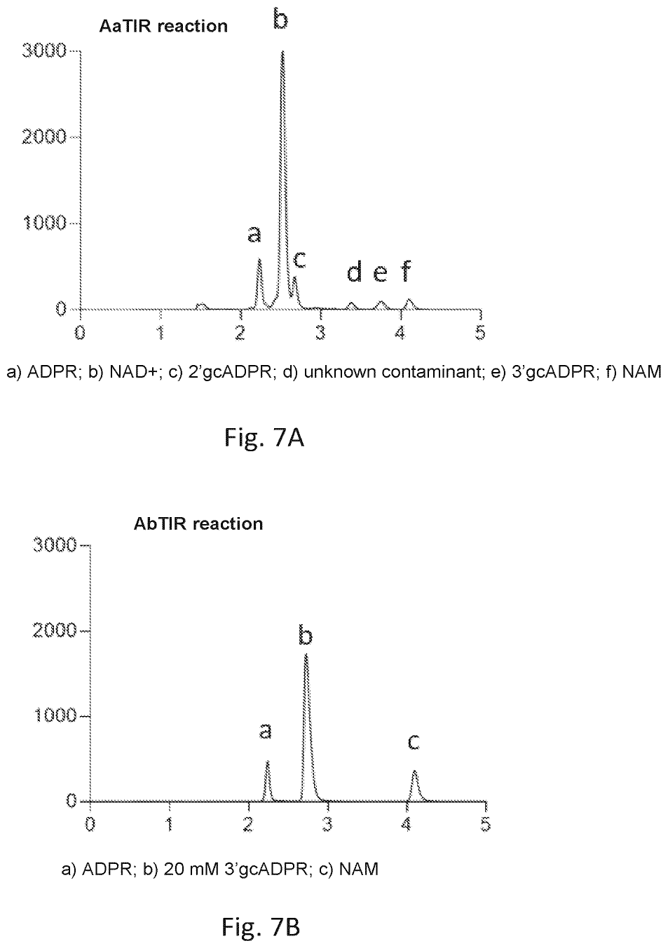

Reactions consisting of 50 mM HEPES-KOH pH 7.5, 150 mM NaCl, 20 mM β-NAD+ (Sigma), and 10 μM AaTIR or AbTIR were incubated at 22° C. for 168 h. Reactions were then filtered using a 3 kDa MWCO centrifugal filter unit and analyzed by HPLC using a running buffer of 97% 50 mM NaH₂PO₄—NaOH pH=8 mixed with 3% acetonitrile at a flow rate of 1 mL min⁻¹ on a C18 column (Zorbax Bonus-RP 4.6×150 mm², 3.5 μm) attached to an Agilent 1200 Infinity Series LC system. HPLC peaks were identified by comparison to chemical standards, and gcADPR peaks were collected by fractionation on the HPLC system (FIGS. 7A, 7B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

ADDIN Mendeley Bibliography CSL_BIBLIOGRAPHY 1. Ofir, G. et al. Antiviral activity of bacterial TIR domains via immune signaling molecules. Nature 600, 116-120 (2021).
2. Burch-Smith, T. M. & Dinesh-Kumar, S. P. The Functions of Plant TIR Domains. Sci. STKE 2007, (2007).
3. Fitzgerald, K. A. & Kagan, J. C. Toll-like Receptors and the Control of Immunity. Cell 180, 1044-1066 (2020).
4. Wan, L. et al. TIR domains of plant immune receptors are NAD+-cleaving enzymes that promote cell death. Science 365, 799-803 (2019).
5. Horsefield, S. et al. NAD+cleavage activity by animal and plant TIR domains in cell death pathways. Science 365, 793-799 (2019).
6. Ka, D., Oh, H., Park, E., Kim, J.-H. & Bae, E. Structural and functional evidence of bacterial antiphage protection by Thoeris defense system via NAD+degradation. Nat. Commun. 11, 2816 (2020).
7. Bayless, A. M. & Nishimura, M. T. Enzymatic Functions for Toll/Interleukin-1 Receptor Domain Proteins in the Plant Immune System. Front. Genet. 11, (2020).
8. Doron, S. et al. Systematic discovery of antiphage defense systems in the microbial pangenome. Science 359, eaar4120 (2018).
9. Peters, J. M. et al. A comprehensive, CRISPR-based functional analysis of essential genes in bacteria. Cell 165, 1493-1506 (2016).
10. Gilchrist, C. L. M. & Chooi, Y.-H. clinker & clustermap.js: automatic generation of gene cluster comparison figures. Bioinformatics 37, 2473-2475 (2021).
11. Chen, I. M. A. et al. IMG/M v.5.0: An integrated data management and comparative analysis system for microbial genomes and microbiomes. Nucleic Acids Res. (2019).
12. Nayfach, S. et al. Metagenomic compendium of 189,680 DNA viruses from the human gut microbiome. Nat. Microbiol. 6, 960-970 (2021).
13. Bondy-Denomy, J. et al. Multiple mechanisms for CRISPR-Cas inhibition by anti-CRISPR proteins. Nature 526, 136-139 (2015).
14. Stanley, S. Y. & Maxwell, K. L. Phage-Encoded Anti-CRISPR Defenses. Annu. Rev. Genet. 52, 445-464 (2018).
15. Li, Y. & Bondy-Denomy, J. Anti-CRISPRs go viral: The infection biology of CRISPR-Cas inhibitors. Cell Host Microbe 29, 704-714 (2021).
16. Athukoralage, J. S. et al. An anti-CRISPR viral ring nuclease subverts type III CRISPR immunity. Nature 577, 572-575 (2020).
17. Tal, N. et al. Cyclic CMP and cyclic UMP mediate bacterial immunity against phages. Cell 184, 5728-5739.e16 (2021).
18. Whiteley, A. T. et al. Bacterial cGAS-like enzymes synthesize diverse nucleotide signals. Nature 567, 194-199 (2019).
19. Cohen, D. et al. Cyclic GMP-AMP signaling protects bacteria against viral infection. Nature 574, 691-695 (2019).
20. Johnson, A. G. et al. Bacterial gasdermins reveal an ancient mechanism of cell death. Science 375, 221-225 (2022).
21. Bernheim, A. et al. Prokaryotic viperins produce diverse antiviral molecules. Nature 589, 120-124 (2021).
22. Morehouse, B. R. et al. STING cyclic dinucleotide sensing originated in bacteria. Nature 586, 429-433 (2020).
23. A. M. Kropinski, A. Mazzocco, T. E. Waddell, E. L. & Johnson, R. P. methods and protocols. in Bacteriophages 69-76 (Humana Press, 2009).
24. Baym, M. et al. Inexpensive multiplexed library preparation for megabase-sized genomes. PLOS One 10, e0128036 (2015).
25. Martin, M. Cutadapt removes adapter sequences from high-throughput sequencing reads. EMBnet.journal 17, 10 (2011).
26. Nurk, S. et al. Assembling Genomes and Mini-metagenomes from Highly Chimeric Reads. in Research in Computational Molecular Biology 158-170 (2013).
27. Hyatt, D. et al. Prodigal: prokaryotic gene recognition and translation initiation site identification. BMC Bioinformatics 11, 119 (2010).
28. A. Mazzocco, T. E. Waddell, E. Lingohr, R. P. J. Enumeration of Bacteriophages Using the Small Drop Plaque Assay System. in Bacteriophages 81-85 (Humana Press, 2009).
29. Steinegger, M. & Söding, J. MMseqs2 enables sensitive protein sequence searching for the analysis of massive data sets. Nat. Biotechnol. 35, 1026-1028 (2017).
30. Deatherage, D. E. & Barrick, J. E. Identification of mutations in laboratory-evolved microbes from next-generation sequencing data using breseq. in Engineering and Analyzing Multicellular Systems 165-188 (Humana Press, 2014).
31. Garb, J. et al. Multiple phage resistance systems inhibit infection via SIR2-dependent NAD&lt;sup&gt;+&lt;/sup&gt; depletion. bioRxiv 2021.12.14.472415 (2021) doi:10.1101/2021.12.14.472415.
32. Katoh, K. & Standley, D. M. MAFFT multiple sequence alignment software version 7: improvements in performance and usability. Mol. Biol. Evol. 30, 772-780 (2013).
33. Nguyen, L.-T., Schmidt, H. A., von Haeseler, A. & Minh, B. Q. IQ-TREE: A fast and effective stochastic algorithm for estimating maximum-likelihood phylogenies. Mol. Biol. Evol. 32, 268-274 (2015).
34. Letunic, I. & Bork, P. Interactive Tree Of Life (iTOL) v5: an online tool for phylogenetic tree display and annotation. Nucleic Acids Res. 49, W293-W296 (2021).
35. Lee, T. S. et al. BglBrick vectors and datasheets: A synthetic biology platform for gene expression. J. Biol. Eng. 5, 12 (2011).
36. Unger, T., Jacobovitch, Y., Dantes, A., Bernheim, R. & Peleg, Y. Applications of the Restriction Free (RF) cloning procedure for molecular manipulations and protein expression. J. Struct. Biol. 172, 34 44 (2010).
37. Frey, S. & Görlich, D. A new set of highly efficient, tag-cleaving proteases for purifying recombinant proteins. J. Chromatogr. A 1337, 95-105 (2014).
38. Peleg, Y. & Unger, T. Application of High-Throughput Methodologies to the Expression of Recombinant Proteins in E. coli. in Structural Proteomics 197-208 (Humana Press, 2008).
39. Zhou, W. et al. Structure of the Human cGAS-DNA Complex Reveals Enhanced Control of Immune Surveillance. Cell 174, 300-311.ell (2018).
40. Eaglesham, J. B., Pan, Y., Kupper, T. S. & Kranzusch, P. J. Viral and metazoan poxins are cGAMP-specific nucleases that restrict cGAS-STING signaling. Nature 566, 259-263 (2019).
41. Liebschner, D. et al. Macromolecular structure determination using X-rays, neutrons and electrons: recent developments in Phenix. Acta Crystallogr. Sect. D, Struct. Biol. 75, 861-877 (2019).
42. Emsley, P. & Cowtan, K. Coot: model-building tools for molecular graphics. Acta Crystallogr. D. Biol. Crystallogr. 60, 2126-32 (2004).
43. Chen, V. B. et al. MolProbity: all-atom structure validation for macromolecular crystallography. Acta Crystallogr. D. Biol. Crystallogr. 66, 12-21 (2010).
44. Karplus, P. A. & Diederichs, K. Linking crystallographic model and data quality. Science 336, 1030-3 (2012).
45. Weiss, M. S. Global indicators of X-ray data quality. J. Appl. Crystallogr. 34, 130-135 (2001).

What is claimed is:

1. A method of treating a disease or disorder associated with a down-regulation of an immune response of a subject comprising administering to the subject a therapeutically effective amount of a gcAPDR molecule, thereby treating the disease or disorder associated with a down-regulation of an immune response of a subject, wherein said gcAPDR molecule comprises at least one of 1′-2′ glycosyl cyclic adenosine diphosphate ribose (1′-2′ gcADPR) and/or 1′-3′ glycosyl cyclic adenosine diphosphate ribose (1′-3′ gcADPR), or a salt, an enantiomer, solvate or hydrate thereof.

2. The method of claim 1, wherein said disease or disorder associated with a down-regulation of the immune response is cancer.

3. The method of claim 2, wherein the cancer is selected from the group consisting of melanoma, breast cancer, pancreatic cancer, prostate cancer, lung cancer, colorectal cancer.

4. The method of claim 1, wherein said gcAPDR molecule is comprised in bacteria.

5. The method of claim 2, wherein said administering comprises intratumoral administration.

6. The method of claim 1, further comprising administering to the subject a therapeutically effective amount of an antibody directed against an immune checkpoint protein.

7. The method of claim 6, wherein said antibody is anti-PDI antibody or an anti-OX40 antibody.

8. An article of manufacture comprising:
(i) 1′-2′ gcADPR and/or 1′-3′ gcADPR, or a salt, an enantiomer, solvate or hydrate thereof; and
(ii) an antibody directed against an immune checkpoint protein.

9. The article of manufacture of claim 8, wherein said 1′-2′ gcADPR and/or said 1′-3′ gcADPR and said antibody are formulated in a single composition.

10. A vaccine comprising a disease associated antigen and an adjuvant comprising 1′-2′ gcADPR and/or 1′-3′ gcADPR. or a salt, an enantiomer, solvate or hydrate thereof.


SEQUENCE LISTING (27 sequences)

SEQ ID NO: 1 - protein, 142 aa, synthetic construct
  REGION 1..142: note = Tad1 from the phage SBSphiJ7
MRELKHELLPTRHTQVFHEDKDKMEFNAPHHFVVVPAGSPLVDQQIHYGRGGNSKKVTVKDFAGKLATVNFQLGPVTEHGANGVMNEDLIAMVITRLQYFQNSEFNCRENAMAITKLEEALMWLNKRTAEREQRGVEGTHEK

SEQ ID NO: 2 - protein, 104 aa, synthetic construct
  REGION 1..104: note = Tad1 polypeptide from Opitutaceae bacterium TAV5
MKILTEGHRYELANFEKKDAPGQVIQFIEKVPESPGSATLVTVNDGTTNEELARVLINRIQHLNGKFPCRENAIAITHFETGLMWLEKRTADRVARNVEGKATT

SEQ ID NO: 3 - protein, 156 aa, synthetic construct
  REGION 1..156: note = Tad1 polypeptide from Opitutaceae bacterium TAV5
MSNQHRILVGHQVNPANDTLTVAVLDRPGRGGACHAYQVSGANMARHPFMRQAVREFAGSDLSDAQVDAAFAQASDGDTSSLILFQDGPIAEAGVNGVTHEVLLAILIDRLEGFQAGPYACEANSKALEHIKYAQQRLLERTRQRMARGMEGTHKV

SEQ ID NO: 4 - protein, 122 aa, synthetic construct
  REGION 1..122: note = Tad1 polypeptide from Clostridioides difficile
MEIKNSLCTQNYTKLYCEDKEEWKYNAPCCFILGRAECEDDCIEPIQYLSFQEGPIKEQGVNGINNEDVILAVITRLEAFQDSPYACRENEKAIEKLEECLMWLRKRTLDRQMRSVEGTSGI

SEQ ID NO: 5 - protein, 119 aa, synthetic construct
  REGION 1..119: note = Tad1 polypeptide from Leptolyngbya sp. PCC 7375
MQKIFEQAYAGTVNRVFVVDEPGPGNGCHQYRTEIEADGAEFCTETNFQKGPVKEVGLNGIQNEHLLAMVIHRLQGFQSGPYSCRENAQALASLQKAMQWMEARTKDRAQRGVEGVNQK

SEQ ID NO: 6 - protein, 132 aa, synthetic construct
  REGION 1..132: note = Tad1 polypeptide from Bacillus cereus
MKRLEHDLLTRKYTEVFHEEEFTNNAPHHFEVYANTGIVMYNPNNVENDNPLPLKVGEVNFQEGPIQEAGINGVMNEDLIAMVICRLEHFNQTDFRCRENSMAITKLEEALLWLRKRTVGREKRGVEGTHTK

SEQ ID NO: 7 - protein, 125 aa, synthetic construct
  REGION 1..125: note = Tad1 polypeptide from Paenibacillus sp. Mc5Re-14
MELVKISNGLLTEKYTTVHHEKDYQFNAPHYFEVHDHSGNVLGSVHFQEGPIQEAGVNGVTNEDLIAMVIARLEGFQNSEFRNRDNAVAITKLEEALLWLRKRTLEREARGVEGTHTKRSGSLCT

SEQ ID NO: 8 - protein, 137 aa, synthetic construct
  REGION 1..137: note = Tad1 polypeptide from Acinetobacter baumannii
MNNSEHETQGHASESIVFGTDIHEDHNGVVVTHNEQIQVENSEKRHELSHGHYYDILSGELVTPIQFQHGPVKEHGVNGITSEALLAILIHRTKILNNNFPCEENKRAITYMENALALFEQRTRDRQQRGVEGLNQA

SEQ ID NO: 9 - protein, 108 aa, synthetic construct
  REGION 1..108: note = Tad1 polypeptide from Clostridium botulinum
LETLHTKVVPGDIEFNSPTSYKIIHKETGKITQNINIQKGPVKENGLNGIFIEDLILICINQLEHFQASKFKCKENESTLRHLYDALATTRSRQYERQLRNVQGKNLK

SEQ ID NO: 10 - protein, 124 aa, synthetic construct
  REGION 1..124: note = Tad1 polypeptide from Clostridium botulinum
MKELSTIQKREKLNTVERIGSEGPGGAYHEYVIKSNSMDSQGNYDVYETIKFQKGARKEEKSQHGVIDSDLLEIVRDRLKSFQAGPFSSRENACALTHVEEALMWMNRRVEDRIERNVLGTNTK

SEQ ID NO: 11 - protein, 122 aa, synthetic construct
  REGION 1..122: note = Tad1 polypeptide from Clostridioides mangenotii
MEIKNGLCTQKYTKVYAEDKEKWKFNAPHHFIVGKADCEDEYIEPIEYVNFQEGPIKEYGINGVNNEDLILMVITRLQAFQDSPYKCRENAMAITKLQECLMWLGKRTLDREVKGIEGTSEI

SEQ ID NO: 12 - protein, 165 aa, synthetic construct
  REGION 1..165: note = Human hSARM1 (residues 560-724 of the full length protein)
MTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 13 - DNA, 315 bp, synthetic construct
  misc_feature 1..315: note = Tad1 polypeptide nucleic acid sequence (Opitutaceae bacterium TAV5)
  source 1..315: mol_type = other DNA
atgaaaatcctcacggaaggacacagatacgaactcgcgaacttcgagaaaaaggacgctcccggccaggtgattcagttcatcgaaaaggttccggagtctccgggttccgcaaccctcgtcaccgtcaatgacgggactaccaatgaggagttggcgcgtgtccttatcaaccgcatccagcacctcaacggcaaattcccctgccgcgaaaacgccatcgccatcacgcacttcgagacggggttgatgtggctcgaaaagcgcacggcggaccgcgtggctcgcaacgtcgaaggcaaggccacgacctaa

SEQ ID NO: 14 - DNA, 471 bp, synthetic construct
  misc_feature 1..471: note = Tad1 polypeptide nucleic acid sequence (Pseudomonas linyingensis)
  source 1..471: mol_type = other DNA
atgagcaatcagcaccgcatccttgtcggtcatcaggtcaacccggccaatgacaccctgaccgtcgccgtcctcgacaggccaggcagaggtggcgcctgccacgcataccaggtctctggcgccaacatggcccgccaccctttcatgcgccaggccgtgcgcgagtttgcgggctctgacctatcggacgcccaggtcgacgccgcctttgcgcaggcctccgacggcgacacttcctcgctgatcctgttccaggatggccccatcgccgaggccggcgtgaacggcgtcacccatgaggtgctgctggcgattctgatcgaccgcctggagggcttccaggctggcccctatgcctgcgaggccaactcaaaggcgctggagcacatcaagtacgcccagcagcgcctccttgagcgcacccgtcagcgtatggcgcgcggcatggaaggaacgcacaaggtctag

SEQ ID NO: 15 - DNA, 369 bp, synthetic construct
  misc_feature 1..369: note = Tad1 polypeptide nucleic acid sequence (Clostridioides difficile)
  source 1..369: mol_type = other DNA
atggagattaaaaatagtttatgcacacaaaattatactaagctttattgcgaggataaagaagaatggaagtataatgcaccatgttgctttatattaggaagggcagaatgtgaggatgattgtatagaaccaattcaatatcttagttttcaagaaggacctataaaagagcagggagttaatggaataaataatgaagatgtaatattagcagttataacaagattagaagcattccaagacagcccatatgcttgtagagaaaatgagaaggctatagagaagttagaagaatgcttgatgtggcttagaaaaagaactctagatagacaaatgagaagtgttgaaggaactagtggtatttaa

SEQ ID NO: 16 - DNA, 360 bp, synthetic construct
  misc_feature 1..360: note = Tad1 polypeptide nucleic acid sequence (Leptolyngbya sp. PCC 7375)
  source 1..360: mol_type = other DNA
atgcaaaagatttttgaacaagcctatgcaggcaccgtcaaccgagttttcgttgttgatgagccagggccaggcaacggatgccatcagtatcgaacagagatagaagcagatggcgctgaattttgcactgagaccaacttccaaaaaggccctgtcaaagaagttggattgaatgggattcagaatgaacatctgctagccatggttatccaccggctgcaagggttccagtctggcccgtactcgtgcagagaaaatgcgcaggcgctagccagtcttcagaaggccatgcagtggatggaagcgagaacgaaagatagggctcaacgtggtgttgaaggagtgaaccagaaatag

SEQ ID NO: 17 - DNA, 399 bp, synthetic construct
  misc_feature 1..399: note = Tad1 polypeptide nucleic acid sequence (Bacillus cereus)
  source 1..399: mol_type = other DNA
atgaaaagactagaacatgatttactaacgagaaagtacactgaggtatttcacgaagaggagtttacaaacaatgctccacatcactttgaagtttatgctaacactggaatcgttatgtacaatcctaataacgtcgagaacgataatccattacctttaaaggttggagaggttaattttcaagaaggaccaatccaagaagctggtattaacggtgtaatgaatgaggatcttatcgctatggttatttgtcgccttgaacatttcaaccaaacagatttccgttgtagagagaactcaatggcaatcactaagttagaagaggcgttactgtggcttagaaaacgaactgtgggtagggaaaaacgaggcgtagaaggtacgcatactaaataa

SEQ ID NO: 18 - DNA, 378 bp, synthetic construct
  misc_feature 1..378: note = Tad1 polypeptide nucleic acid sequence (Paenibacillus sp. Mc5Re-14)
  source 1..378: mol_type = other DNA
atggagctggtaaagatatcaaatggacttcttaccgaaaaatatactactgtccatcacgaaaaggattatcaattcaacgcaccacactattttgaggttcatgatcatagcggtaacgtcctgggcagtgttcatttccaagaaggaccgatccaagaagctggagtaaatggagtgaccaacgaagatttaattgctatggttattgctcgtctggaaggattccaaaacagtgagttccgtaatcgggataatgcagttgcaatcacaaagttggaagaagcgcttctgtggcttagaaaacggacattagaacgcgaagcccgaggcgtggaaggaacacataccaaaaggtcgggttcattatgcacctga

SEQ ID NO: 19 - DNA, 414 bp, synthetic construct
  misc_feature 1..414: note = Tad1 polypeptide nucleic acid sequence (Acinetobacter baumannii)
  source 1..414: mol_type = other DNA
atgaacaattctgaacacgaaactcaaggccatgcgtcagaaagtatagtttttggcacagacattcatgaagatcataatggcgttgtagtaacgcacaatgagcagattcaagttgagaattcagaaaaacgtcatgaattaagtcatggtcattattacgatattttgtctggtgagttagttacaccaattcaatttcaacatggtccagttaaggaacatggggtgaatggtataactagtgaagcgcttttagccattttgattcatcggactaaaattttgaacaataactttccatgtgaagaaaataagcgagcgataacttacatggaaaatgcactagcactttttgaacagcgtacacgtgatcgacaacaacgtggtgttgagggcttaaatcaagcctaa

SEQ ID NO: 20 - DNA, 327 bp, synthetic construct
  misc_feature 1..327: note = Tad1 polypeptide nucleic acid sequence (Clostridium botulinum)
  source 1..327: mol_type = other DNA
ttggaaacattgcataccaaagttgttccaggagatatagaatttaattctcctacatcttataaaataattcataaagaaactggtaagattactcaaaatataaatatacaaaaaggacctgttaaggaaaatgggttaaatggaatttttattgaagacttaatattaatttgtataaaccaattagaacattttcaagctagtaaatttaaatgtaaagaaaatgaatctacgctaaggcatttatatgatgcattagctacaactagatcaagacaatatgaaagacaattaagaaatgtacaaggtaaaaacttaaaataa

SEQ ID NO: 21 - DNA, 375 bp, synthetic construct
  misc_feature 1..375: note = Tad1 polypeptide nucleic acid sequence (Clostridium botulinum)
  source 1..375: mol_type = other DNA
atgaaagaattaagtacaattcaaaaaagagagaaattaaacactgttgaaagaataggaagcgaaggtccaggaggtgcatatcatgaatatgtaattaaaagtaattctatggatagtcaagggaattatgatgtttatgaaactatcaagttccaaaaaggtgctagaaaagaagagaaatcacaacacggtgtaatagattctgacttattagaaatagtaagagatagattaaagagttttcaagctggaccattctcaagtagagaaaatgcttgtgctttaacacatgttgaagaagctttaatgtggatgaatagaagggtagaggacagaattgaaagaaatgtattaggtacaaatactaagtag

SEQ ID NO: 22 - DNA, 369 bp, synthetic construct
  misc_feature 1..369: note = Tad1 polypeptide nucleic acid sequence (Clostridioides mangenotii)
  source 1..369: mol_type = other DNA
atggaaattaaaaacggtttatgcacacagaaatatacaaaggtatatgcagaggataaagaaaaatggaagtttaatgcaccacatcattttattgttggtaaagcagattgcgaggatgaatatatagaaccaattgaatatgttaactttcaagaaggtccaataaaagagtacggaataaatggagtaaataacgaagatttaattctcatggttataacaaggctacaagctttccaagatagtccttacaaatgcagagagaacgctatggctataactaaactacaagagtgcttaatgtggttaggaaaaagaacgctagacagagaggttaaaggaattgaaggcacaagcgagatctaa

SEQ ID NO: 23 - DNA, 429 bp, synthetic construct
  misc_feature 1..429: note = DNA sequence of Tad 1 from the phage SBSphiJ7
  source 1..429: mol_type = other DNA
atgagggaattaaaacatgagttactgcccaccagacatacacaggtgttccatgaagacaaagataaaatggagttcaacgcacctcatcatttcgtagtggttcctgccggaagtcctttagtcgatcagcagattcactatggaagaggcggaaatagcaagaaggttaccgtaaaagactttgcaggtaaattggctacagtaaactttcagctaggtccagttacagagcatggagccaatggggttatgaatgaggacctcatcgcaatggttattactcgcttacagtattttcagaatagtgaatttaattgccgggagaacgctatggcaattacaaaacttgaagaggcgcttatgtggcttaataaacgtacagcagaacgagaacagcgtggagtagaaggaactcacgagaagtaa

SEQ ID NO: 24 - protein, 100 aa, synthetic construct
  REGION 1..100: note = ThsA from B.cereus MSX-D12
MNPIVELFIKDFTKEVMEENAAIFAGAGLSMSVGYVSWAKLLEPIAQEIGLDVNKENDLVSLAQYYCNENQGNRGRINQIILDEFSRKVDLTENHKILAR

SEQ ID NO: 25 - protein, 192 aa, synthetic construct
  REGION 1..192: note = ThsB protein from B cereus MSX-D12
MAKRVFFSFHYQDVIDFRVNVVRNHWVTKLNQSAAGVFDASLWEDAKKTSDIALKRLINGGLNNTSVTCVLIGSQTFNRRWVRYEIMKSIEKGNKIIGIHINAFKDKYGNIKSKGPNPFDYLGYQYSSDGKQLHLYEWTGGKWEEYKDLAPYRVNQIAPESLRGKFYSLSSVYRVYDWVADDGYNKFSSWVN

SEQ ID NO: 26 - protein, 224 aa, synthetic construct
  REGION 1..224: note = Brachypodium distachyon (BdTIR)
MASSGLSSRRSIMASRLTASAEAVNEPRRGAVVSRRVEYDEESLAGAGGESRYEVFINHRGVDTKRTVARLLYDRLAQAGLRGFLDNMSMRPGDRLEERIGSAIRECTVAVAIFSPSYCDSEYCLRELAMLVESRKAIIPIFYDIKPSDLLLPQAVADSEVYLPRDLERFKFALREAKHTVGITYDSATGDMAELVSAAADAVMYNMEKMETVQRRETMILSRL

SEQ ID NO: 27 - protein, 159 aa, synthetic construct
  REGION 1..159: note = Drosophila dSARM1 (residues 807-967 of the full length protein
MAKTLDVFVSYRRSNGSQLASLLKVHLQLRGFSVFIDVERLEAGKFDNGLLNSIRQAKNFVLVLTPDALHRCINDEDCKDWVHREIVAALNSNCNIIPIIDQQFDWPEVERLPEDMRSVAHFNGVNWIHDYQDACIDKLERFLRGEKNIDRIAAMVPGT